(19) United States
(12) Patent Application Publication
(10) Pub. No.: US 2025/0041381 A1
(43) Pub. Date: Feb. 6, 2025

(54) Prophylactic Uses of Theta Defensins

(71) Applicant: The University of Southern California, Los Angeles, CA (US)

(72) Inventors: Michael E. Selsted, Pasadena, CA (US); Justin B. Schaal, Orange, CA (US); Patti Tran, Alhambra, CA (US); Dat Q. Tran, Alhambra, CA (US)

(21) Appl. No.: 18/706,707

(22) PCT Filed: Nov. 1, 2022
(86) PCT No.: PCT/US2022/048569

§ 371 Date: May 1, 2024

Related U.S. Application Data
(60) Provisional application No. 63/274,889, filed on Nov. 2, 2021.

(51) Int. Cl. A61K 38/17

(52) U.S. Cl. CPC A61K 38/1729

(57) ABSTRACT

Compositions and methods that provide safe and effective prophylactic treatment of a wide range of microbial pathogens are described. Such compositions and methods incorporate θ defensins and analogs of θ defensins, which have broad antimicrobial effects, have been found to maintain effective concentrations in the blood stream for extended periods of time, and are safe to administer at high doses.

### CROSS REFERENCE TO RELATED APPLICATIONS

This application is a national stage application of International Patent Application No. PCT/US2022/048569, filed on Nov. 1, 2022, which claims the benefit and priority of U.S. Provisional Patent Application No. 63/274,889 filed on Nov. 2, 2021. These and all other referenced extrinsic materials are incorporated herein by reference in their entirety. Where a definition or use of a term in a reference that is incorporated by reference is inconsistent or contrary to the definition of that term provided herein, the definition of that term provided herein is deemed to be controlling.

This invention was made with government support under AI125141 and AR068833 awarded by the National Institutes of Health. The government has certain rights in the invention.

### REFERENCE TO SEQUENCE LISTING

A computer readable XML file entitled “103388.0015PCT-Sequence-Listing”, that was created on Oct. 27, 2022, and is 29 kb in size, contains the sequence listing for this application, has been filed with this application, and is hereby incorporated by reference in its entirety.

### FIELD OF THE INVENTION

The field of the invention is prophylactic treatment of microbial infections.

### BACKGROUND

The background description includes information that may be useful in understanding the present invention. It is not an admission that any of the information provided herein is prior art or relevant to the presently claimed invention, or that any publication specifically or implicitly referenced is prior art.

Prophylactic treatment for prevention of communicable disease is a fundamental part of public healthcare policy. Such treatments are provided in the absence of active disease.

Accordingly, considerations in prophylaxis include the risk of side effects of the prophylactic treatment exceeding the risk of acquiring and being negatively impacted by the disease that it is seeking to prevent. Accordingly, in order to be useful a prophylactic treatment should both be effective and provide a margin of safety at effective doses that exceeds that of treatments used to address active, symptomatic disease.

Vaccines are well known examples of prophylactic therapies. Immunization with a suitable vaccine elicits an antibody response to a specific pathogen in the vaccinated individual. Current vaccine formulations are highly effective and very low risk. For example, currently over 400 million doses of vaccine directed to the causative agent of COVID-19 (SARS-CoV-2), which is highly effective (approximately 94% for mRNA-based formulations) in reducing transmission and apparently prevents serious illness in rare break-through infections, have been administered in the United States. Close monitoring by the Vaccine Adverse Event Reporting System, however, reports only about a 0.0021% death rate from all causes following immunization.

Such prophylactic vaccines, however, are generally highly specific. As a result, there is inevitably a lag between recognition of a new pathogen and development, testing, manufacturing, and distribution of a safe and effective vaccine directed to it. While newer approaches, such as the use of mRNA vaccines, have reduced this lag period it is still significant.

In addition, individuals with poor immune responses are less likely to develop an antibody response that is sufficient to prevent or reduce the severity of infection following immunization.

Such at-risk individuals may require additional rounds of immunization, or may fail to develop a protective antibody response altogether.

Thus, there is still a need for safe prophylactic compositions and methods that provide effective protection against a broad range of pathogens and is effective in immunocompromised individuals.

### SUMMARY OF THE INVENTION

The inventive subject matter provides compositions and methods in which 6-defensins and functional analogs of 6-defensins, which have been found to have a wide range of antimicrobial activities, to persist in the body following administration, and to be safe at high dosages, are used to provide prophylactic protection against microbial pathogens.

Embodiments of the inventive concept include methods of providing prophylactic treatment for a microbial infection by identifying an individual that is in need of prophylactic treatment for the microbial infection and administering a 0-defensin (e.g., RTD-1 (SEQ ID NO. 1)) or analog of a 0-defensin to the individual using a protocol that maintains at least 50% of peak plasma concentration of the 0-defensin or θ-defensin analog for at least 4 hours following administration. Initially, an effective θ-defensin or θ-defensin analog is identified that maintains at least 50% of peak plasma concentration for at least 4 hours following administration. Following identification of an individual that is in need of prophylactic treatment, the effective θ-defensin or θ-defensin analog is administered to the individual in an amount sufficient that at least 50% of peak plasma concentration is sufficient to exert antimicrobial against the microbe against which prophylaxis is desired. In some embodiments such an individual is identified as a high risk individual, such as a neonate, a premature infant, a child with an under or partially developed immune system, an elderly individual, as recovering from surgery, as recovering from accidental injury, as having cancer, as immunocompromised, as having undergone organ, bone marrow, or stem cell transplantation, individuals receiving or recovering from chemotherapy, as receiving or recovering from radiotherapy, as receiving or recovering from immunotherapy, as receiving or recovering from immunosuppression therapy, as having underlying heart disease and/or damage, as having underlying kidney disease and/or damage, as having underlying liver disease and/or damage, as having underlying lung disease and/or damage, as having obesity, and/or is identified on the basis of exposure to others that are within a distance over which the microbe can be transmitted. The microbe against which such methods are directed can be is a virus (e.g., SARS-CoV-2), bacteria, fungus, or protozoan. In such methods a θ-defensin or θ-defensin analog is administered at up to 15 mg/kg. The θ-defensin or θ-defensin analog can be administered as a single dose, such as a dose sufficient to provide the θ-defensin or θ-defensin analog to the individual in need of treatment at from 0.1 mg/kg to 15 mg/kg. Alternatively, the θ-defensin or θ-defensin analog can be administered as two or more doses (e.g., at a frequency of once every 12 hours to once every 48 hours), where each individual dose is sufficient to provide the θ-defensin or θ-defensin analog to the individual in need of treatment at from 0.1 mg/kg to 15 mg/kg.

Embodiments include formulations for providing prophylactic treatment of microbial infections that include one or more effective θ-defensin(s) and/or effective θ-defensin analog(s), as well as use of one or more effective θ-defensin(s) and/or effective θ-defensin analog(s) in preparing such formulations.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIGS. 1A to 1D: FIGS. 1A to 1D show typical results of plasma concentration vs. time studies in different species. FIG. 1A depicts typical mean (SD) plasma concentration-time profiles of RTD-1 (SEQ ID NO. 1) in mice. (n=4/time point). FIG. 1B depicts typical mean (SD) plasma concentration-time profiles of RTD-1 (SEQ ID NO. 1) in rats (n=6/time point).

FIG. 1C depicts typical mean (SD) plasma concentration-time profiles of RTD-1 (SEQ ID NO. 1) in cynomolgus monkeys (n=2-12/dosing group). FIG. 1D depicts typical mean (SD) plasma concentration-time profiles of RTD-1 (SEQ ID NO. 1) in vervet (n=1) following single dose IV administration.

FIGS. 2A and 2B: FIG. 2A depicts typical mean (SD) plasma concentration versus time profiles in rats following a 7-day repeat once daily i.v. administrations of 5 mg/kg/day. FIG. 2B depicts typical mean (SD) plasma concentration versus time profiles in rats following a 7-day repeat once daily i.v. administrations of 10 mg/kg/day.

FIGS. 3A to 3D: FIG. 3A depicts typical mean (SD) plasma concentration versus time profiles in cynomolgus monkeys following repeated i.v. administrations of RTD-1 (SEQ ID NO. 1) at 5 mg/kg/day in the recovery group (n=4). FIG. 3B depicts typical mean (SD) plasma concentration versus time profiles in cynomolgus monkeys following repeated i.v. administrations of RTD-1 (SEQ ID NO. 1) at 10 mg/kg/day in the recovery group (n=4). FIG. 3C depicts typical mean (SD) plasma concentration versus time profiles in cynomolgus monkeys following repeated i.v. administrations of RTD-1 (SEQ ID NO. 1) at 15 mg/kg/day in the recovery group (n=4). FIG. 3D depicts typical mean (SD) plasma concentration versus time profiles in cynomolgus monkeys following repeated i.v. administrations of RTD-1 (SEQ ID NO. 1) at 15 mg/kg in the recovery group (n=4).

FIGS. 4A and 4B: FIG. 4A shows typical results of an assessment of dose proportionality of Cmax in cynomolgus monkeys following a single dose administration of RTD-1 (SEQ ID NO. 1) (0.3, 1, 3, 10, or 15 mg/kg). FIG. 4B shows typical results of an assessment of dose proportionality of AUC_{0-∞} in cynomolgus monkeys following a single dose administration of RTD-1 (SEQ ID NO. 1) (0.3, 1, 3, 10, or 15 mg/kg).

FIGS. 5A and 5B: FIG. 5A depicts typical results of interspecies allometric correlation studies. The plot was created based on the results of RTD-1 (SEQ ID NO. 1) single-dose PK studies. The dashed line represents the predicted for CL (6.44 L/h) for a70 kg adult. FIG. 5B depicts typical results of interspecies allometric correlation studies. The plot was created based on the results of RTD-1 (SEQ ID NO. 1) single-dose PK studies. The dashed line represents the predicted for V_{SS} (28.0 L) for a 70 kg adult.

FIG. 6: FIG. 6 depicts typical mean (SD) density and distribution of ¹⁴C-RTD-1 (SEQ ID NO. 1) in tissues and organs 1 hour and 24 hours after intravenous administration in female rats

### DETAILED DESCRIPTION

The inventive subject matter provides compositions and methods that provide safe and effective prophylactic treatment of a wide range of microbial pathogens. Inventors have found that theta defensins can maintain effective concentrations in the blood stream for extended periods of time, and are safe to administer at high doses.

One should appreciate that the disclosed techniques provide many advantageous technical effects including provision of safe and effective prevention of microbial infections in high-risk populations without the need for development of organism-specific formulations.

The following description includes information that may be useful in understanding the present invention. It is not an admission that any of the information provided herein is prior art or relevant to the presently claimed invention, or that any publication specifically or implicitly referenced is prior art.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

The following discussion provides many example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

Inventors have found that theta defensins (e.g., RTD-1 (SEQ ID NO. 1)) are extremely well tolerated and show favorable pharmacokinetics with rapid distribution and a prolonged (e.g., in excess of 2, 3, 4, 5, 6, 8, 12, 23, 36, 48 hours or longer) retention of effective concentrations following administration indicative of a prolonged effect useful in prophylactic treatment. Similarly, studies show that administration of theta defensin in large amounts (e.g., up to 15 mg/kg or more) is well tolerated and indicated a safety suitable for prophylactic use, particularly in vulnerable and high-risk individuals.

In view of the activity of theta defensins against a wide range of microbial (e.g., viral, bacterial, fungal, protozoan) infections, Inventors believe that such theta defensins can be useful in prophylactic treatment of such microbial infections. In view of the high degree of tolerance observed Inventors believe that such prophylactic treatment has particular utility in high-risk populations that are particularly susceptible to such infections. Such high risk populations include, but are not limited to, neonates, premature infants, children with under or partially developed immune systems, elderly individuals, individuals recovering from surgery, individuals recovering from accidental injury, individuals with cancer, immunocompromised individuals, individuals who have undergone organ, bone marrow, or stem cell transplantation, individuals receiving or recovering from chemotherapy, individuals receiving or recovering from radiotherapy, individuals receiving or recovering from immunotherapy, individuals with underlying heart disease and/or damage, individuals with underlying kidney disease and/or damage, individuals with underlying liver disease and/or damage, individuals with underlying lung disease and/or damage, and obese individuals.

In other embodiments, a high-risk individual can be a person who is at particularly high risk of exposure to a microbial pathogen, for example by being within a distance of potential sources of infection (e.g., symptomatic or asymptomatic infected persons) over which the microbe can be transmitted. This distance can vary with the microbe and its mode of transmission. For example, such a distance can range from direct or intimate contact to about 2 meters or more (for aerosol transmission) from a source of the pathogen. Such high-risk individuals can, for example, be in a profession that requires contact with or proximity to potentially infectious individuals (e.g., a health care provider, a teacher, an emergency responder, law enforcement, etc.). Alternatively, a high-risk individual can be one that anticipates a social interaction that can potentially involve contact with or exposure to an infectious person.

While the theta defensin RTD-1 (SEQ ID NO. 1) is cited herein, inventors contemplate that other θ-defensins can be effective in methods of the inventive concept. Within the context of this application an effective θ-defensin is one that shows a microbicidal and/or microbistatic (i.e., halting microbial reproduction) effect at concentrations attainable in a human being following administration by oral administration, injection, infusion, inhalation, and/or application to an ocular or mucus membrane. Such effects can be demonstrated against viral, bacterial, fungal, and/or protozoan pathogens. Similarly, methods of the inventive concept can utilize an effective amount of an effective θ-defensin. Within the context of this application, an effective amount is an amount of an effective θ-defensin that is sufficient to maintain a microbicidal and/or microbistatic effect in an individual to which the effective amount of the effective θ-defensin is administered for a period of 4 hours, 8 hours, 12 hours, 24 hours, 48 hours, 3 days, 1 week, 10 days, 2 weeks 3 weeks, one month, or more than one month following administration. In some embodiments the effective amount of the effective θ-defensin or effective θ-defensin analog can be 10%, 20%, 30%, 40%, 50%, or more of peak serum concentration following administration.

Examples of suitable θ-defensins include RTD-1 (SEQ ID NO. 1), RTD-2 (SEQ ID NO. 2), RTD-3 (SEQ ID NO. 3), RTD-4 (SEQ ID NO. 4), RTD-5 (SEQ ID NO. 5), RTD-6 (SEQ ID NO. 6), BTD-1 (SEQ ID NO. 7), BTD-2 (SEQ ID NO. 8), BTD-3 (SEQ ID NO. 9), BTD-4 (SEQ ID NO. 10), BTD-5 (SEQ ID NO. 11), BTD-6 (SEQ ID NO. 12), BTD-7 (SEQ ID NO. 13), BTD-8 (SEQ ID NO. 14), BTD-9 (SEQ ID NO. 15), and/or BTD-10 (SEQ ID NO. 16). Peptides derived from alternative splicing of the human proto-defensin HTDp (SEQ ID NO. 17), which is transcribed but not translated in nature, are also considered. In some embodiments two or more θ-defensins can be used in methods of the inventive concept.

In place of or in addition to naturally occurring θ-defensins, embodiments of the inventive concept can employ one or more effective θ-defensin analogs. The term θ-defensin analog refers to a cyclic peptide having about 40%, 50%, 60%, 70%, 80%, 90% or greater sequence identity with a native θ-defensin peptide sequence. A θ-defensin analog can incorporate one, two, three, or more core features of a native θ-defensin. Exemplary core features include cyclic structure, the presence of one, two, three, or more disulfide bonds within the peptide (e.g., between pairs of cysteines of the analog), having a positive charge when in solution under physiological conditions, and the presence of beta pleated sheet secondary structure. Such θ-defensin analogs can include 10, 11, 12, 13, 14, 15, 16, 17, 18, 19, 20, or more than 20 amino acids, and in some embodiments can incorporate non-naturally occurring amino acids. An analog of a θ-defensin can include one or more L-amino acid(s), one or more D-amino acid(s), and/or a mixture of L- and D-amino acids. In some embodiments non-peptide bonds can be utilized between adjacent amino acid residues of a θ-defensin analog. θ-defensin analogs can represent one or more deletion or substitution of amino acids of a native θ-defensin sequence. Such substitutions can be conservative (e.g., where the substituted amino acid(s) retain(s) charge, hydrophobicity, hydrophilicity, and/or steric properties of the native amino acid). In some embodiments θ-defensin analogs can include grafting or conjugation of non-peptide moieties, for example polyethylene glycol and/or other hydrophilic polymers, cell-receptor targeting moieties, and/or moieties that aid in processing/purification.

Within the context of this application an effective θ-defensin analog is one that shows a microbicidal and/or microbistatic (i.e., halting microbial reproduction) effect at concentrations attainable in a human being following administration by oral administration, injection, infusion, inhalation, and/or application to an ocular or mucus membrane. Such effects can be demonstrated against viral, bacterial, fungal, and/or protozoan pathogens. Similarly, methods of the inventive concept can utilize an effective amount of an effective θ-defensin analog. Within the context of this application, an effective amount is an amount of an effective θ-defensin analog that is sufficient to maintain a microbicidal and/or microbistatic effect in an individual to which the effective amount of the effective θ-defensin is administered for a period of 12 hours, 24 hours, 48 hours, 3 days, 1 week, 10 days, 2 weeks 3 weeks, one month, or more than one month following administration.

Examples of suitable θ-defensin analogs include peptide 1 (SEQ ID NO. 18), peptide 2 (SEQ ID NO. 19), peptide 3 (SEQ ID NO. 20), peptide 4 (SEQ ID NO. 21), peptide 5 (SEQ ID NO. 22), peptide 6 (SEQ ID NO. 23), peptide 7 (SEQ ID NO. 24), peptide 8 (SEQ ID NO. 25), peptide 9 (SEQ ID NO. 26), peptide 10 (SEQ ID NO. 27), peptide 11 (SEQ ID NO. 28), peptide 12 (SEQ ID NO. 29), peptide 13 (SEQ ID NO. 30), peptide 14 (SEQ ID NO. 31), and peptide 15 (SEQ ID NO. 32). In some embodiments two or more effective θ-defensin analogs can be used in methods of the inventive concept. In some embodiments a combination of one or more effective θ-defensin(s) and one or more effective θ-defensin analog(s) can be used.

It should be appreciated that θ-defensins have very similar sizes, secondary and tertiary structures, charges, degrees of hydrophobicity, and resistance to proteolysis. Accordingly, it is reasonable to expect that θ-defensins have similar degradation and excretion pathways following administration. Similarly, θ-defensin analogs as described herein retain essentially structural, charge, and degrees of hydrophobicity as naturally occurring θ-defensins and it is reasonable to expect that similar degradation and excretion pathways are utilized. As such, suitable θ-defensins and/or θ-defensin analogs that are effective to provide pharmacokinetic properties equivalent to or better than RTD-1 (SEQ ID NO. 1) are readily identifiable, for example through animal studies as described below and/or by reference. Similarly, suitable θ-defensins and/or θ-defensin analogs that are effective to antimicrobial effects equivalent to or better than RTD-1 (SEQ ID NO. 1) are readily identifiable, for example through animal models of microbial disease and/or by reference. In some embodiments an effective θ-defensin and/or θ-defensin analog can have similar or improved pharmacokinetic properties and safety profiles relative to RTD-1 (SEQ ID NO. 1), and so can be used for prophylactic treatment of microbial infection. Similarly, while intravenous administration is described herein other routes of administration can also be suitable. Suitable alternative routes for administration include subcutaneous injection, intraperitoneal injection, injection into cerebrospinal fluid, topical administration, inhalation, and/or ingestion.

In a typical method of prophylactic treatment, a θ-defensin and/or θ-defensin analog that is effective at providing antimicrobial activity at plasma concentrations that are sustained for at least 4 hours (i.e., an effective θ-defensin and/or θ-defensin analog) is identified. This can be accomplished by experimentation (for example, using animal models of microbial infection and/or pharmacokinetic studies), or can be determined from historical data. Similarly, an individual in need of prophylactic therapy is identified and an effective θ-defensin and/or θ-defensin analog administered. Such an effective dose can provide a plasma concentration of the effective θ-defensin and/or θ-defensin analog that is sufficient to exert a microbicidal and/or microbistatic effect for at least 4 hours, 8 hours, 12 hours, 24 hours, 36 hours, 48 hours, three days, a week, two weeks, three weeks, a month, two months, or more. Such a method can, for example, provide an effective θ-defensin and/or θ-defensin analog that maintains at least 50% of the peak plasma concentration for at least 4 hours following administration to a person that has been identified as needing prophylactic treatment to prevent microbial infection, where the dose of sufficient that 50% of the peak plasma concentration of the effective θ-defensin and/or θ-defensin analog exerts an antimicrobial effect against the target microbe. Suitable routes of administration include injection (e.g., subcutaneous, intramuscular, or peritoneal injection), inhalation, and/or topical (e.g., to a mucus membrane) administration.

Compounds useful for prophylactic therapy should be considered very safe for use. Safety and pharmacokinetic parameters of RTD-1 (SEQ ID NO. 1), which can be considered a prototypical θ-defensin, are provided below. Single and multiple-dose studies performed in rats and cynomolgus monkeys demonstrated the excellent safety profile of intravenous RTD-1 (SEQ ID NO. 1) administration. Repeat administration of RTD-1 (SEQ ID NO. 1) was well tolerated in rats at doses up to 10 mg/kg, and therefore, the NOAEL in rats was established at 10 mg/kg/day. Treatment-related mortality and adverse clinical signs were observed in rats treated at 20 mg/kg, including cold to touch, abnormal body color, inability to walk, extreme dehydration, and tremors. In cynomolgus monkeys, single and repeated daily dose administration of RTD-1 (SEQ ID NO. 1) was tolerated up to 15 mg/kg/day, with no major treatment-related adverse findings or toxicities. Given the lack of adverse findings, the NOAEL was established at 15 mg/kg/day in cynomolgus monkeys. The NOAEL in the cynomolgus monkeys was established at a higher dose when compared with the rats, demonstrating that RTD-1 (SEQ ID NO. 1) was better tolerated in cynomolgus monkeys. Most changes noted in hematological, serum chemistry, and coagulation parameters in both rats and cynomolgus monkeys were determined as non-adverse due to their low magnitude, lack of consistency between the two species, lack of dose dependence, and/or reversibility by the end of the recovery period. Therefore, these data demonstrate the safety of intravenous RTD-1 (SEQ ID NO. 1) at a dose up to 10 mg/kg/day in rats and 15 mg/kg/day in cynomolgus monkeys.

The PK of intravenous RTD-1 (SEQ ID NO. 1) following single and multiple ascending doses in multiple species is characterized by extensive tissue distribution and prolonged elimination. The volume of distribution (V_{SS}) normalized to a bodyweight of the animals receiving 5 mg/kg of RTD-1 (SEQ ID NO. 1) varied across species, with 1,048, 1,461, 550 mL/kg, in mice, rats, and cynomolgus monkeys, respectively. The relatively large V_{SS} indicates that RTD-1 (SEQ ID NO. 1) extensively distributes to tissues. The biodistribution study confirmed extensive tissue distribution, particularly in the liver. The prolonged elimination half-life observed in cynomolgus monkeys in the recovery group (47.2 h) is suggestive of tissue redistribution.

Analysis of the single- and multiple ascending dose studies in rats and monkeys showed a greater than dose-proportional increase in AUC0-∞ and Cmax suggestive of nonlinear PK. Several therapeutic proteins exhibit nonlinear PK mediated by different mechanisms. For instance, exenatide and recombinant human interferon (IFN) (e.g., IFN-β1a) display nonlinear kinetics due to the saturation of the elimination pathway, such as target-mediated drug disposition (TMDD) (24, 25). Alternatively, nonlinear PK of cyclosporin A and erythropoietin were attributable to the saturation of tissue binding and receptors in target tissues, respectively (26, 27). The widespread distribution of ¹⁴C-RTD-1 (SEQ ID NO. 1) in rats in the biodistribution study, which could explain the large V_{SS} estimated in the preclinical PK studies, suggests that saturation of the peptide within these tissues may be one potential source of nonlinearity. Since the greatest accumulation of RTD-1 (SEQ ID NO. 1) occurred in the liver and kidney, a dose-dependent decrease in CL observed in rats and cynomolgus monkeys could also explain the nonlinearity. Consistent with data from other small peptides (<10 kDa) which are predominately cleared through the glomerular filtration, appreciable amounts of ¹⁴C-RTD-1 (SEQ ID NO. 1) were recovered from the urine (accounting for 7% at 24 h). In addition, a relatively significant portion was recovered in feces (accounting for 4% at 24 h) indicating that elimination of RTD-1 (SEQ ID NO. 1) occurs through renal and biliary excretion (28, 29). Therefore, the nonlinear PK of RTD-1 (SEQ ID NO. 1) may be attributable to saturation of uptake and/or efflux transporters present in the liver or kidney. However, the definitive role of hepatic/renal transporters in the distribution and elimination of RTD-1 (SEQ ID NO. 1) requires further investigation.

Interspecies allometric scaling provides methods for extrapolating PK data from preclinical species to humans and is commonly used to predict an appropriate dosage for FIH clinical trials. Since RTD-1 (SEQ ID NO. 1) is believed to follow linear PK at the HED for efficacy, as evidenced by dose-proportional increases in the AUC0-∞ at lower doses in cynomolgus monkeys (0.3-3 mg/kg), we performed interspecies allometric scaling using simple allometry to predict human PK. For macromolecules that are renally excreted, human CL can be adequately predicted using the simple allometric equation (30). As three or more preclinical species are typically needed to reliably scale the parameters to humans, available single-dose data from mice and vervet were included in the analysis (31). The estimated allometric scaling exponents of 0.829 and 0.866 for CL and V_{SS} respectively, agree with the values reported for other therapeutic proteins, which are 0.65-0.84 for CL and 0.84-1.02 for V_{SS} (32). The target AUC was previously established in a mouse model of LPS-induced ALI, where a single subcutaneous injection of 5- or 25 mg/kg RTD-1 (SEQ ID NO. 1), which resulted in mean AUC0-∞ of 3,869 and 9,001 ng*h/mL respectively, led to a significant decrease in airway neutrophil burden and inflammatory cytokines/chemokines without mortality (3). The HED of 0.3 mg/kg daily was determined using the predicted human CL (6.44 L/h) from interspecies allometric scaling and the target AUC for efficacy (from the murine model of endotoxin-induced ALI). Based on the FDA's recommendation for a 10-fold safety factor, the predicted first-in-human (FIH) dose in a clinical trial is approximately 0.03 mg/kg for an adult (33, 34). This approximation of the dose for the FIH study is predicted to be well below the NOAEL established by preclinical animals and therefore projected to be safe in humans. The HED equivalent to NOAEL in cynomolgus monkeys of 15.9 mg/kg was higher than both the HED equivalent to NOAEL and LOAEL in rats (3.1 and 11.7 mg/kg, respectively). Since cynomolgus monkeys are more physiologically similar to humans than rats, doses up to 15.9 mg/kg may be tolerated in humans. Furthermore, this indicates that the HED required for efficacy (0.36-0.83 mg/kg) is approximately 19- to 45-fold lower than the HED calculated based on NOAEL in cynomolgus monkeys, further ensuring safety in humans.

It should be appreciated that simple allometry may not account for dose-dependent (nonlinear) processes. However, since nonlinearity was more evident at higher dose ranges, Applicants believe that at the doses selected for FIH clinical trial, RTD-1 (SEQ ID NO. 1) is predicted to exhibit linear PK. It should also be appreciated that the target AUC_{0-∞} used to determine the FIH dose was established in a murine model of LPS-induced ALI and therefore may not reflect the actual target AUC required for efficacy in humans. Inventors, however, believe this is an appropriate animal model to derive the target AUC for efficacy in prophylactic treatment of a wide range of microbial infections.

### Pharmacokinetics

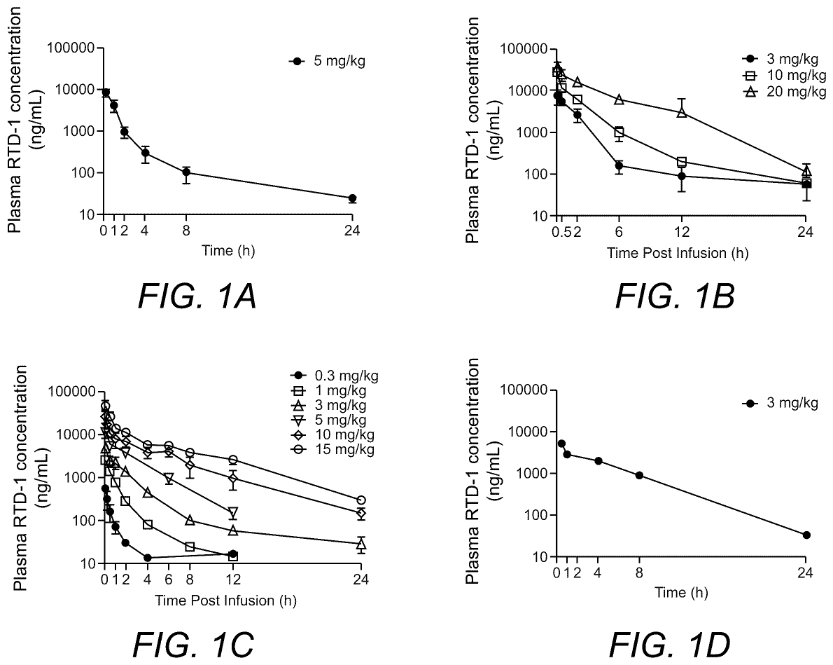

Mouse studies: The mean plasma concentration-time profile after single-dose administration of RTD-1 (SEQ ID NO. 1) in mice is shown in FIG. 1A, and the corresponding pharmacokinetic (PK) parameters calculated by non-compartmental analysis (NCA) are listed in Table 1. Due to terminal blood collection from each mouse, a pooled PK result was generated. After single i.v. bolus administration, RTD-1 (SEQ ID NO. 1) displayed a biphasic profile, with a relatively short distribution phase followed by a longer elimination phase with a half-life of 6.05 hours.

TABLE 1
Single dose pharmacokinetics in mice receiving
RTD-1 (SEQ ID NO. 1) 5 mg/kg

| Parameters | Estimate (SE) |
|---|---|
| Cₘₐₓ (ng/mL) | 8,919 (1,088) |
| λz (h⁻¹) | 0.114 |
| AUCτ (ng*h/mL) | 12,282 |
| AUC_{0-∞} (ng*h/mL) | 12,497 |
| CL (mL/h/kg) | 400 |
| MRT (h) | 2.62 |
| Vss (mL/kg) | 1,048 |

Cₘₐₓ, maximum observed plasma concentration; λz, terminal elimination rate constant; AUCτ, area under the curve to dosing interval (24 h), AUC_{0-∞}, area under the curve extrapolated to infinity; CL, clearance; MRT, mean residence time; Vss, volume of distribution at steady state

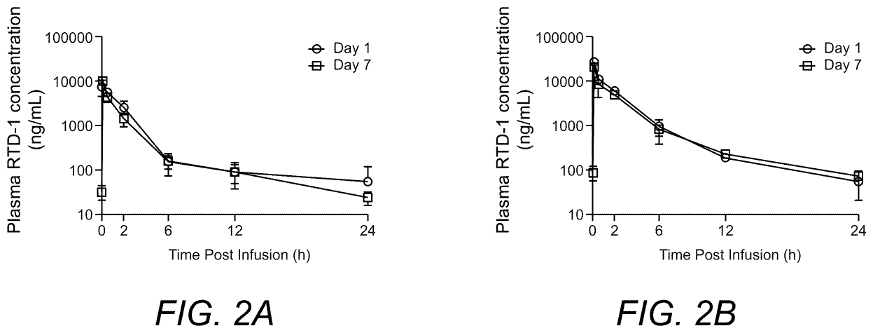

Rat studies: The mean plasma concentration-time profiles in rats following single (5, 10, or 20 mg/kg) or multiple doses (5 or 10 mg/kg/day) administrations of RTD-1 (SEQ ID NO. 1) are depicted in FIG. 1B and FIG. 2, respectively, and the corresponding PK parameters are summarized in Table 2. Of the total 180 infusions from 36 rats, five infusions deviated more than 10% from the intended 20-min infusion. However, these deviations did not occur on blood PK sampling days and therefore did not influence the PK analysis. Due to sparsely sampled data per rat, a pooled PK result was generated in WinNonlin. Plasma levels of RTD-1 (SEQ ID NO. 1) were undetectable during the recovery period (Day 25) in all rats, except for one female rat that received 10 mg/kg, which had a concentration of 12.5 ng/mL. Early removal of the rats in the 20 mg/kg group precluded the PK analysis with repeat dosing at this dose level. The Cmax was slightly higher in females compared to male rats, but the differences did not reach statistical significance. While both Cmax and AUC_{0-∞} appeared to increase proportionally to the dose based on 95% confidence interval (CI) of the slope including 1, (Cmax, Y=1.110*X+8.936 [Slope 95% CI: 0.7066 to 1.513], R2=0.6803; AUC0-∞, Y=1.543*X+9.448 [Slope 95% CI: 0.4460 to 2.639], R2=0.9969), a comprehensive analysis of dose proportionality in rats was limited due to pooled calculations of AUC0-∞ at each dose level, and the relatively narrow range of doses tested. The AUCτ on Day 7 was slightly lower (22% and 19% for the 5- and 10 mg/kg groups, respectively) when compared with their AUC0-∞ on Day 1, indicating no significant drug accumulation.

TABLE 2
Single and multiple-dose pharmacokinetics of intravenous RTD-1 (SEQ ID NO. 1)

| | 5 mg/kg | | 10 mg/kg | | 20 mg/kg |
|---|---|---|---|---|---|
| Parameters | Day 1 | Day 7 | Day 1 | Day 7 | Day 1 |
| Cₘₐₓ | 7,468 (1,202)*^{, #} | 10,137 (688) | 27,533 (1,419)* | 21,850 (759) | 35,400 (5,350)^{#} |
| (ng/mL) | | | | | |
| λz (h⁻¹) | 0.056 | 0.104 | 0.148 | 0.133 | 0.221 |
| AUCτ | 14,956 | 12,468 | 41,669 | 34,121 | 126,860 |
| (ng*h/mL) | | | | | |
| AUC_{0-∞} | 15,948 | 12,704 | 42,085 | 34,688 | 127,386 |
| (ng*h/mL) | | | | | |
| CL (mL/h/kg) | 334 | 401 | 240 | 293 | 157 |
| MRT (h) | 4.37 | 2.92 | 2.88 | 3.45 | 4.81 |
| Vss (mL/kg) | 1,461 | 1,173 | 691 | 1,011 | 758 |

(Estimate, SE)
Cₘₐₓ, maximum observed plasma concentration; λz, terminal elimination rate constant; AUCτ, area under the curve to dosing interval (24 h), AUC_{0-∞}, area under the curve extrapolated to infinity; CL, clearance; MRT, mean residence time; Vss, volume of distribution at steady state
*^{, #}denote statistically significant differences between matching groups (p < 0.05)

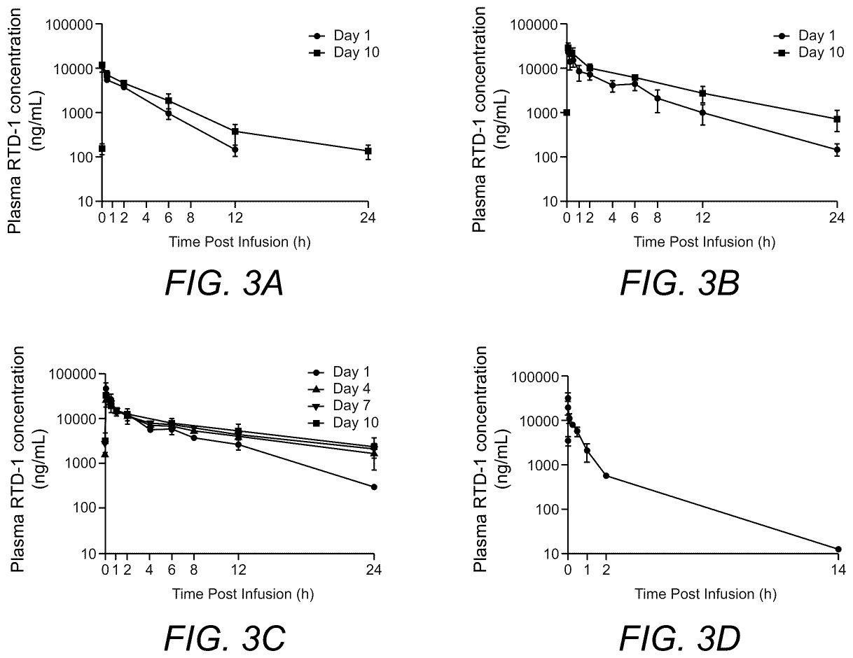

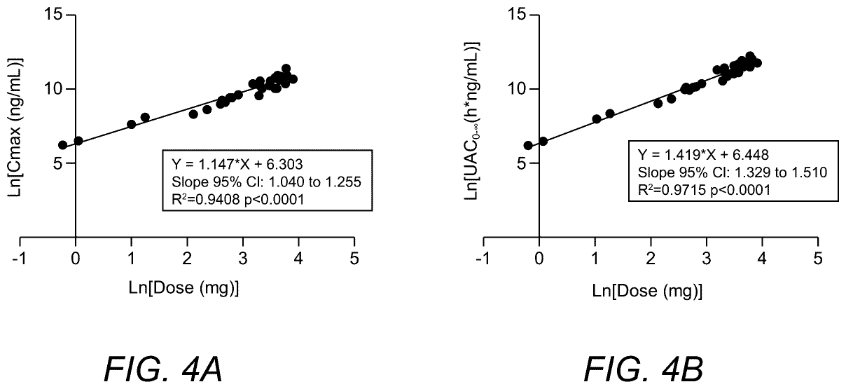

Cynomolgus monkey studies: The mean plasma concentration-time profiles in cynomolgus monkeys following single or multiple dose administrations of RTD-1 (SEQ ID NO. 1) are illustrated in FIG. 1C and FIG. 3, respectively. The corresponding PK parameters calculated by NCA are outlined in Table 3. Of the total 233 infusions from 24 cynomolgus monkeys, four infusions deviated more than 10% from the intended 1-h infusion. However, these deviations did not occur on blood PK sampling days and therefore did not influence the PK analysis. Data from two separate studies involving single and multiple dosing in cynomolgus monkeys were combined in this analysis. Overall, concentration-time profiles displayed a biphasic pattern, with a prolonged elimination phase at higher doses. Plasma concentrations of RTD-1 (SEQ ID NO. 1) after a single dose of 0.3 or 1 mg/kg were detectable up to 12 h post end of infusion. In the GLP-compliant 10-day TK study, all animals received ten i.v. doses except for one female monkey in 15 mg/kg, which received a total of 9 doses due to issues with venous access. Extended sampling with cynomolgus monkeys assigned to the recovery group (15 mg/kg) revealed that plasma levels of RTD-1 (SEQ ID NO. 1) were quantifiable on Day 12 (537 ng/mL) and Day 24 (12.5 ng/mL), indicating that RTD-1 (SEQ ID NO. 1) exhibits a long terminal half-life (FIG. 3D). The average terminal half-life in the recovery animals was 47.2 h when compared with 9.53 h in the main group with the shorter sampling period. The Cmax and AUC0-∞ were slightly higher in males compared to females, but the differences did not reach statistical significance. Detailed assessment of dose proportionality in cynomolgus monkeys administered a single i.v. dose ranging from 0.3 to 15 mg/kg revealed that both Cmax and AUC0-∞ increased greater than dose-proportional (FIG. 4). Specifically, AUC_{0-∞} was dose-proportional at lower doses (0.3-3 mg/kg) but began to deviate from dose proportionality at higher doses (>5 mg/kg) (data not shown).

Dose proportionality assessment at steady-state demonstrated that while the Cmax increased dose proportionally (Cmax, Y=0.9553*X+6.805 [Slope 95% CI: 0.5573 to 1.353, R2=0.5705]), the AUCτ increased greater than dose proportionally (AUCτ, Y=1.422*X+6.745 [Slope 95% CI: 1.072 to 1.772, R²=0.7916]). However, these results were limited due to the narrow range of doses examined. Comparisons of AUCs after a single dose (Day 1) and repeat dose administrations (Day 10) revealed statistically significant accumulations at 5- and 10 mg/kg, yielding approximately 1.4- and 1.5-fold higher mean AUCτ compared to the mean AUC0-∞ on Day 1 for 5- and 10 mg/kg, respectively [5 mg/kg (p=0.0229) and 10 mg/kg (p=0.0103)]. Although there was a trend towards RTD-1 (SEQ ID NO. 1) accumulation at 15 mg/kg at a steady-state (Day 10), the difference did not reach statistical significance (p=0.1879). Statistical analysis was not performed with the PK parameters calculated on Days 4 and 7 in the 15 mg/kg group due to the small number of animals (n=2).

TABLE 3
Single and multiple-dose pharmacokinetics of intravenous RTD-1 (SEQ ID NO. 1) in cynomolgus monkeys

| | 0.3 | 1 | 3 | 5 | | 10 | | 15 | | | |
|---|---|---|---|---|---|---|---|---|---|---|---|
| | mg/kg | mg/kg | mg/kg | mg/kg | | mg/kg | | mg/kg | | | |
| | Day 1 | Day 1 | Day 1 | Day 1 | Day 10 | Day 1 | Day 10 | Day 1 | Day 4 | Day 7 | Day 10 |
| Parameters | n = 2 | n = 2 | n = 2 | n = 6 | n = 6 | n = 8 | n = 6 | n = 12 | n = 2 | n = 2 | n = 9 |
| Cₘₐₓ | 582 | 2,560 | 4,750 | 10,700 | 11,675 | 26,675 | 27,333 | 46,458 | 31,100 | 32,850 | 32,233 |
| (ng/mL) | (109) | (820) | (905) | (2,455) | (2,036) | (8,240) | (7,361) | (15,247)^{†} | (283) | (919) | (13,933)^{†} |
| λz (h⁻¹) | 0.523 | 0.251 | 0.078 | 0.323 | 0.157 | 0.192 | 0.120 | 0.149 | 0.072 | 0.071 | 0.078 |
| | (0.119) | (0.035) | (0.017) | (0.016)^{#} | (0.009)^{#} | (0.034)* | (0.018)* | (0.019)^{†} | (0.020) | (0.017) | (0.020)^{†} |
| AUCτ | 591 | 3,818 | 10,334 | 27,160 | 38,936 | 76,083 | 117,529 | 138,135 | 139,595 | 154,149 | 173,315 |
| (ng*h/mL) | (145) | (961) | (2,239) | (4,286) | (9,829) | (23,562) | (26,077) | (34,942) | (2,366) | (12,406) | (67,934) |
| AUC_{0-∞} | 617 | 3,875 | 10,740 | 27,176 | 39,997 | 76,820 | 124,790 | 142,095 | 167,061 | 187,609 | 212,728 |
| (ng*h/mL) | (137) | (973) | (2,485) | (4,292) | (10,219) | (23,871) | (30,604) | (35,842) | (16,034) | (31,623) | (104,487) |
| CL | 498 | 266 | 287 | 188 | 136 | 143 | 88.5 | 114 | 107 | 97.6 | 103 |
| (mL/h/kg) | (111) | (66.9) | (66.4) | (29.2)^{#} | (36.5)^{#} | (48.2)* | (18.5)* | (25.7) | (1.82) | (7.86) | (53.0) |
| MRT_{inf} | 1.24 | 1.82 | 4.30 | 2.94 | 4.84 | 4.40 | 7.20 | 5.48 | 11.8 | 12.3 | 12.0 |
| (h) | (0.16) | (0.07) | (0.60) | (0.15) | (0.57) | (0.50) | (1.04) | (0.76) | (2.46) | (3.08) | (3.33) |
| Vss | 626 | 486 | 1,215 | 550 | 645 | 629 | 624 | 629 | 1,263 | 1,193 | 1,139 |
| (mL/kg) | (218) | (139) | (112) | (74.6) | (123) | (231) | (88.2) | (176)^{†} | (242) | (204) | (411)^{†} |

Mean (SD)
Cₘₐₓ, maximum observed plasma concentration;
λz, terminal elimination rate constant;
AUCτ, area under the curve to dosing interval (24 h),
AUC_{0-∞}, area under the curve extrapolated to infinity;
CL, clearance;
MRT, mean residence time;
Vss, volume of distribution at steady state
^{#}, *, ^{†}denote statistically significant differences within the dosing group between Day 1 and Day 10 (p < 0.05)

Vervet monkey studies: The mean plasma concentration-time profile of RTD-1 (SEQ ID NO. 1) in the vervet monkeys after a single i.v. bolus administration of RTD-1 (SEQ ID NO. 1) is shown in FIG. 1D, and the PK parameters are presented in Table 4. Following the bolus administration, plasma concentrations of RTD-1 (SEQ ID NO. 1) declined monoexponentially.

However, the plasma concentrations collected after 24 h were below the lower limit of quantification and therefore excluded from the analysis.

TABLE 4
Pharmacokinetics of single dose RTD-1
(SEQ ID NO. 1) (3 mg/kg) in vervet

| Parameters | Estimate |
|---|---|
| Cₘₐₓ (ng/mL) | 5,193 |
| λz (h⁻¹) | 0.204 |
| AUCτ (ng*h/mL) | 21,949 |
| AUC_{0-∞} (ng*h/mL) | 22,110 |
| CL (mL/h/kg) | 136 |
| MRT (h) | 4.71 |
| Vss (mL/kg) | 639 |

Cₘₐₓ, maximum observed plasma concentration; λz, terminal elimination rate constant; AUCτ, area under the curve to dosing interval (24 h), AUC_{0-∞}, area under the curve extrapolated to infinity; CL, clearance; MRT, mean residence time; Vss, volume of distribution at steady state

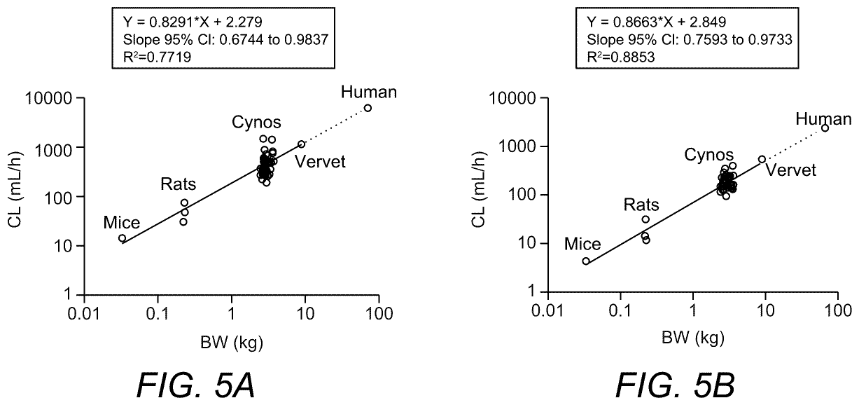

Interspecies allometric scaling: Overall, linear regression of logarithmically transformed CL or V_{SS} against log-transformed body weight (BW) from the four preclinical species resulted in a reasonable fit, as evidenced by the relatively high r2. (FIG. 5). The allometric scaling equations for CL and V_{SS} were Y=190-1·BW^{0.8291} (r2=0.7719) and Y=706.3·BW^{0.8663} (r2=0.8853), respectively, which yielded the predicted human CL of 6.44 L/h and volume of distribution at steady state (V_{SS}) of 28.0 L. Based on the target plasma AUC0-∞ of approximately 3,869 and 9,001 ng*h/mL, which were previously established in a murine model of endotoxin-induced acute lung injury (ALI), the estimated human equivalent doses (HED) to reach therapeutic efficacy are between 24.9 and 58.0 mg for a 70 kg individual, or 0.36 and 0.83 mg/kg.

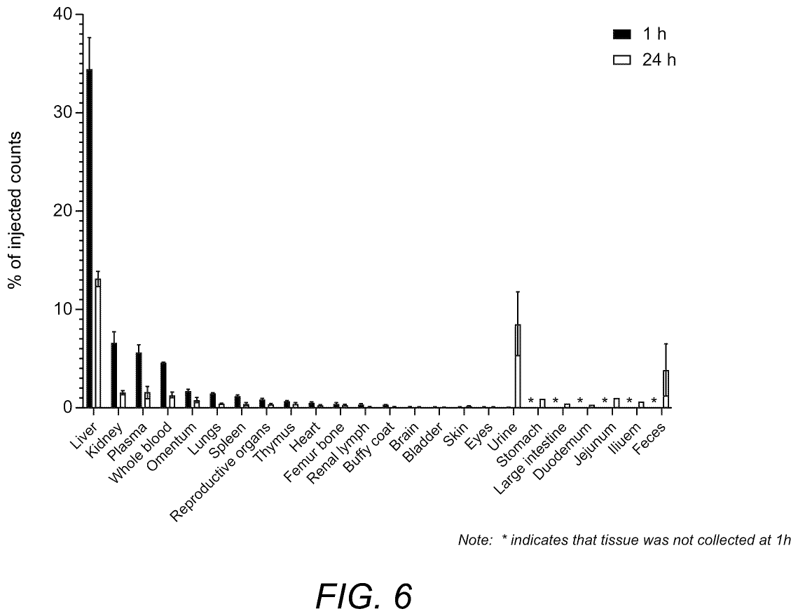

Biodistribution: A biodistribution study was undertaken to determine the patterns of distribution and potential routes of elimination of RTD-1 (SEQ ID NO. 1) in rats after a single dose i.v. administration of ¹⁴C-RTD-1 (SEQ ID NO. 1) equivalent to 5 mg/kg. Widespread distribution of ¹⁴C-RTD-1 (SEQ ID NO. 1) was observed at 1 h, with the highest density measured in the liver, followed by the kidney (FIG. 6). At 1 h, there was a trace amount of ¹⁴C counts measured in the urine, skin, leg muscle, eyes, and brain. After 24 h, the density of ¹⁴C counts in the tissues and organs decreased compared to counts detected at 1 h, except for in the urine. The ¹⁴C counts in urine increased from trace amounts at 1 h to 8.5% after 24 h. Moreover, approximately 4% of the ¹⁴C-RTD-1 (SEQ ID NO. 1) dose administered was recovered in the feces at 24 h, suggesting that the major route of elimination is urinary, followed by biliary excretion.

### Safety

Rat studies: The results of the safety assessments of RTD-1 (SEQ ID NO. 1) administration are provided in detail in the supplementary materials. In general, single doses up to 10 mg/kg were well-tolerated in male and female rats. During the study, mortality was observed in a total of 7 rats. Specifically, 1 out of 18 female rats in the placebo group was found dead on Day 6, and another 1 out of 16 female rats in the 5 mg/kg dose group was found dead on Day 15 of the study. However, the mortality of the female rat in the 5 mg/kg group was determined to be unrelated to RTD-1 (SEQ ID NO. 1) treatment due to minimal clinical manifestations until the day of death and the timing of the event. Additionally, a single i.v. administration of 20 mg/kg RTD-1 (SEQ ID NO. 1) led to acute, treatment-related mortality in 5 out of 12 rats on Day 1. Of the five rats, three male rats were found dead, and one male and one female rat were euthanized due to moribund conditions.

Following once daily i.v. infusion of RTD-1 (SEQ ID NO. 1) (5- and 10 mg/kg/day), non-adverse, treatment-related clinical signs such as muscle fasciculation, lethargy, swollen nose, chin, and/or cheeks, swollen front limbs, reluctance to walk and/or stand, hypoactivity, ataxia, and increased respiration, were noted throughout the study at both dose levels, but were temporary and resolved during the recovery period. There were no significant changes in the body weight in rats except for those in the 20 mg/kg dosing group, where significant decreases in body weights were recorded in both male and female rats (data not shown). Food consumption was not significantly affected by treatment administration in rats at any dose level. Due to the unexpected mortality and adverse treatment-related clinical observations at 20 mg/kg, the study in this dosing group was prematurely terminated, and the remaining rats were euthanized before their scheduled administration on Days 1 or 2. Adverse clinical observations associated with mortalities included cold to touch, laying on the side, abnormal body color, inability to walk, extreme dehydration, tremors, and labored respiration.

No treatment-related changes in hematological parameters were observed in male and female rats at 5 mg/kg at the end of treatment (Day 8) (Table 5). At the end of recovery, RBC volume distribution width (RDW) was significantly elevated in female rats at 5 mg/kg and was outside of the historical control range (HCR) for female Sprague Dawley rats (data not shown). At 10 mg/kg, a significant decrease in absolute reticulocytes in male rats treated with 10 mg/kg at the end of treatment. However, this value was within the HCR for male Sprague Dawley rats of this age and therefore was considered non-adverse (18). In female rats, there were significant increases in WBC, and absolute lymphocytes and monocytes at the end of treatment when compared to controls. However, these increases were considered non-adverse due to lack of dose-dependency and the reversibility of the changes by the end of the recovery period (data not shown). At the end of recovery, mean cell hemoglobin (MCH) and mean cell hemoglobin concentration (MCHC) increased modestly in male rats compared to the controls, but the values remained within the reference range for male rats of similar age. At 20 mg/kg, white blood cell counts (WBC), relative and absolute neutrophils, relative and absolute monocytes, and relative and absolute large unclassified cells (LUC) counts were significantly elevated in male rats, while relative lymphocytes, relative and absolute eosinophils, and platelet count significantly decreased on Day 2 compared to the controls at the end of treatment. The relative and absolute neutrophil, relative lymphocyte, and relative and absolute monocyte values were outside of the HCR for male rats. In female rats, there were significant increases in absolute reticulocytes and monocytes, while significant decreases in relative and absolute eosinophils were observed on Day 2 (interim euthanasia) when compared with controls at the end of treatment. Due to the premature termination of the study in the 20 mg/kg group, the reversibility of the alterations in these parameters could not be determined. However, regardless of statistical significance, these changes in female rats were considered non-adverse as the values were within the HCR for female rats.

TABLE 5
Hematological results of male and female rats at the end of treatment

| | Male | | | | Female | | | |
|---|---|---|---|---|---|---|---|---|
| | 0 | 5 | 10 | 20 | 0 | 5 | 10 | 20 |
| Parameter | mg/kg | mg/kg | mg/kg | mg/kg^{d} | mg/kg | mg/kg | mg/kg | mg/kg^{d} |
| WBC (10³/mm³) | 7.95 | 8.40 | 10.05 | 13.70 | 6.95 | 6.50 | 10.75 | 8.15 |
| | (1.93) | (2.53) | (3.28) | (4.90) ^{a, b} | (2.43) | (3.50) | (3.68) ^{a} | (5.70) |
| Hemoglobin (g/dL) | 13.8 | 14.1 | 14.2 | 12.1 | 13.2 | 13.5 | 13.7 | 15.2 |
| | (0.9) | (1.0) | (0.7) | (1.0) ^{b, c} | (0.9) | (1.0) | (0.4) | (4.6) |
| Hct (%) | 42.6 | 43.8 | 43.7 | 37.3 | 40.8 | 40.5 | 41.2 | 46.9 |
| | (2.9) | (1.7) | (1.2) | (3.7) ^{b, c} | (2.2) | (1.9) | (1.8) | (14.3) |
| RBC (10⁶/mm³) | 6.69 | 7.06 | 7.06 | 6.04 | 6.92 | 6.88 | 7.21 | 7.63 |
| | (0.64) | (0.23) | (0.38) | (0.47) ^{b, c} | (0.36) | (0.33) | (0.40) | (2.19) |
| MCH (pg) | 20.4 | 20.0 | 20.2 | 20.0 | 19.3 | 19.1 | 19.0 | 19.6 |
| | (0.7) | (1.4) | (0.6) | (0.6) | (0.8) | (1.0) | (0.5) | (0.9) |
| MCV (fL) | 62.4 | 61.8 | 62.6 | 62.5 | 58.0 | 57.9 | 57.3 | 61.0 |
| | (3.1) | (2.9) | (3.1) | (2.1) | (2.7) | (2.8) | (2.4) | (2.2) ^{c} |
| MCHC (g/dL) | 32.0 | 32.1 | 32.4 | 31.8 | 33.1 | 33.4 | 33.3 | 32.3 |
| | (0.8) | (0.3) | (0.7) | (0.5) | (0.9) | (0.4) | (0.4) | (0.3) ^{b, c} |
| RDW (%) | 14.1 | 14.0 | 13.1 | 14.3 | 12.5 | 12.7 | 13.0 | 12.2 |
| | (1.5) | (1.2) | (1.1) | (1.2) | (0.3) | (0.8) | (0.4) | (0.6) ^{c} |
| HDW (g/dL) | 2.27 | 2.30 | 2.20 | 2.44 | 2.37 | 2.37 | 2.45 | 2.37 |
| | (0.29) | (0.09) | (0.20) | (0.25) ^{c} | (0.19) | (0.16) | (0.16) | (0.32) |
| Neutrophils (%) | 17.4 | 20.2 | 22.6 | 61.1 | 22.9 | 26.7 | 19.9 | 33.6 |
| | (6.0) | (4.7) | (7.9) | (6.1) ^{a, b, c} | (16.0) | (11.1) | (5.5) | (14.5) |
| Lymphocytes (%) | 75.6 | 71.8 | 70.9 | 30.9 | 69.8 | 68.1 | 72.0 | 60.5 |
| | (7.8) | (5.4) | (11.2) | (4.7) ^{a, b, c} | (18.6) | (8.7) | (4.4) | (12.9) |
| Monocytes (%) | 2.5 | 2.9 | 2.7 | 8.1 | 3.1 | 2.6 | 3.5 | 3.8 |
| | (1.3) | (1.6) | (1.1) | (2.6) ^{a, b, c} | (1.3) | (1.0) | (1.4) | (1.5) |
| Eosinophils (%) | 3.3 | 3.9 | 3.0 | 1.1 | 4.1 | 3.6 | 3.3 | 1.5 |
| | (0.5) | (1.3) | (1.9) | (0.3) ^{a, b, c} | (2.5) | (2.4) | (1.0) | (0.5) ^{a, b, c} |
| Basophils (%) | 0.3 | 0.3 | 0.3 | 0.2 | 0.3 | 0.3 | 0.4 | 0.5 |
| | (0.1) | (0.1) | (0.2) | (0.1) | (0.2) | (0.2) | (0.2) | (0.5) |
| LUC (%) | 0.3 | 0.3 | 0.3 | 0.5 | 0.3 | 0.3 | 0.35 | 0.5 |
| | (0.2) | (0.1) | (0.2) | (0.5) ^{a} | (0.1) | (0.1) | (0.28) | (0.3) |
| Reticulocytes (%) | 5.8 | 5.2 | 4.6 | 6.4 | 4.0 | 3.9 | 4.1 | 5.3 |
| | (1.4) | (0.4) | (0.8) | (1.6) ^{b, c} | (1.2) | (1.0) | (0.6) | (1.4) |
| Neutrophil absolute | 1.48 | 2.04 | 1.85 | 8.40 | 1.75 | 1.33 | 2.18 | 2.55 |
| (10³/mm³) | (0.83) | (0.96) | (0.61) | (3.42) ^{a, b, c} | (0.77) | (0.99) | (1.30) | (2.72) |
| Lymphocyte absolute | 5.49 | 6.03 | 6.69 | 4.24 | 4.40 | 4.78 | 7.13 | 5.56 |
| (10³/mm³) | (2.00) | (1.81) | (2.92) | (1.79) ^{b, c} | (1.75) | (3.21) | (1.76) ^{a} | (2.93) |
| Monocyte absolute | 0.19 | 0.22 | 0.28 | 0.93 | 0.17 | 0.17 | 0.39 | 0.33 |
| (10³/mm³) | (0.16) | (0.20) | (0.10) | (0.29) ^{a, b, c} | (0.09) | (0.08) | (0.13) ^{a, b} | (0.26) ^{a} |
| Eosinophils absolute | 0.27 | 0.37 | 0.31 | 0.15 | 0.28 | 0.30 | 0.33 | 0.13 |
| (10³/mm³) | (0.05) | (0.10) | (0.24) | (0.06) ^{a, b, c} | (0.22) | (0.23) | (0.14) | (0.06) ^{a, c} |
| Basophils absolute | 0.02 | 0.03 | 0.03 | 0.03 | 0.02 | 0.02 | 0.04 | 0.06 |
| (10³/mm³) | (0.01) | (0.02) | (0.02) | (0.03) | (0.03) | (0.02) | (0.02) | (0.06) |
| LUC absolute | 0.02 | 0.03 | 0.03 | 0.07 | 0.02 | 0.02 | 0.04 | 0.05 |
| (10³/mm³) | (0.01) | (0.02) | (0.02) | (0.05) ^{a, b, c} | (0.03) | (0.03) | (0.03) | (0.03) |
| Reticulocytes | 378.3 | 359.7 | 314.2 | 373.3 | 277.8 | 256.7 | 277.3 | 326.0 |
| absolute (10³/mm³) | (90.6) | (38.0) | (33.4) ^{a} | (106.5) ^{c} | (60.6) | (53.5) | (42.0) | (60.4) ^{a, b} |
| Platelets (10³/mm³) | 895 | 1,052 | 1,086 | 728 | 822 | 801 | 939 | 604 |
| | (93) | (145) | (192) | (86) ^{a, b, c} | (169) | (306) | (315) | (524) ^{c} |

Data represents median (IQR)
WBC, White blood cell count; RBC, Red blood cell count; RDW, RBC volume distribution width; HDW, Hemoglobin concentration distribution width; MCH, Mean cell hemoglobin; MCHC, Mean cell hemoglobin concentration; MCV, Mean cell volume; LUC, Large unclassified Table 5cells.
^{a} p < 0.05 compared with control (0 mg/kg)
^{b} p < 0.05 compared with 5 mg/kg
^{c} p < 0.05 compared with 10 mg/kg
^{d}Measurement on day of unscheduled euthanasia (Day 2), excludes animals found dead

There were no significant treatment-induced abnormalities in serum biochemical parameters in male and female rats that received 5 or 10 mg/kg/day at the end of treatment (Table 6). At the end of recovery, glucose levels were slightly elevated in female rats at 10 mg/kg (data not shown). At the highest dose examined (20 mg/kg), chloride and albumin levels significantly decreased while serum urea nitrogen (Urea N) levels increased in male rats on Day 2 compared to controls. In female rats, alanine aminotransferase (ALT), calcium, and gamma-glutamyl transferase (GGT) levels were significantly elevated compared to controls. In both male and female rats, albumin/globulin ratio (A/G), creatinine, glucose, triglyceride levels were significantly elevated while total protein (TP), sodium (Na), and globulin levels significantly reduced when compared with controls. Albumin, A/G, globulin, Urea N, TP, and triglyceride levels were outside the HCR in male rats and A/G, GGT, globulin, glucose, TP, and triglycerides were outside the HCR in female rats. The remaining parameters were within the HCR and therefore considered non-adverse.

TABLE 6
Serum biochemical data of male and female rats at the end of treatment

| | Male | | | | Female | | | |
|---|---|---|---|---|---|---|---|---|
| | 0 | 5 | 10 | 20 | 0 | 5 | 10 | 20 |
| Parameter | mg/kg | mg/kg | mg/kg | mg/kg^{d} | mg/kg | mg/kg | mg/kg | mg/kg^{d} |
| ALT (U/L) | 42.5 | 40.0 | 32.5 | 74.0 | 33.0 | 35.0 | 32.5 | 62.0 |
| | (5.5) | (9.8) | (4.5) | (38.0) ^{b, c} | (8.0) | (7.0) | (6.0) | (28.5) ^{a, b, c} |
| Albumin (g/dL) | 3.7 | 3.8 | 3.7 | 2.7 | 3.6 | 3.9 | 3.9 | 3.0 |
| | (0.2) | (0.0) | (0.2) | (0.0) ^{a, b, c} | (0.2) | (0.3) | (0.1) | (0.6) ^{b, c} |
| A/G | 2.4 | 2.2 | 2.35 | 2.70 | 2.20 | 2.10 | 2.15 | 3.00 |
| | (0.4) | (0.3) | (0.18) | (0.10) ^{a, b, c} | (0.10) | (0.20) | (0.18) | (0.40) ^{a, b, c} |
| ALP (U/L) | 248.5 | 212.0 | 212.5 | 232.0 | 124.0 | 92.0 | 109.5 | 142.0 |
| | (72.8) | (40.3) | (51.5) | (45.0) | (28.0) | (21.0) | (30.5) | (34.5) ^{b, c} |
| AST (U/L) | 158.5 | 133.0 | 113.5 | 245.0 | 149.0 | 139.0 | 122.0 | 397.0 |
| | (45.8) | (34.0) | (16.8) | (100.0) ^{b, c} | (22.0) | (35.0) | (14.3) | (345.5) ^{b, c} |
| Calcium (mg/dL) | 9.60 | 9.75 | 9.70 | 9.40 | 9.60 | 9.80 | 9.95 | 10.30 |
| | (0.18) | (0.45) | (0.20) | (0.50) ^{b} | (0.20) | (0.20) | (0.28) | (0.60) ^{a, b} |
| Chloride | 105.0 | 104.5 | 105.0 | 101.0 | 105.0 | 105.0 | 105.0 | 104.0 |
| (mmol/L) | (1.8) | (1.8) | (1.0) | (1.0) ^{a, b, c} | (3.0) | (2.0) | (2.0) | (3.0) |
| Cholesterol | 59.5 | 70.5 | 66.0 | 67.0 | 52.0 | 55.0 | 55.0 | 52.0 |
| (mg/dL) | (13.0) | (10.3) | (14.3) | (7.0) | (7.0) | (15.0) | (26.5) | (6.5) |
| CK (U/L) | 828 | 766 | 613 | 707 | 914 | 792 | 474 | 1,506 |
| | (342) | (396) | (255) | (615) | (450) | (198) | (211) | (2,605) |
| Creatinine | 0.2 | 0.2 | 0.3 | 0.4 | 0.3 | 0.3 | 0.3 | 0.5 |
| (mg/dL) | (0.0) | (0.1) | (0.1) | (0.1) ^{a, b, c} | (0.0) | (0.0) | (0.0) | (0.2) ^{a, b, c} |
| GGT (U/L) | 0.0 | 0.0 | 0.0 | 0.0 | 0.0 | 0.0 | 0.0 | 4.0 |
| | (0.0) | (0.0) | (0.0) | (0.0) | (0.0) | (0.0) | (0.0) | (5.5) ^{a, b, c} |
| Globulin (g/dL) | 1.6 | 1.7 | 1.6 | 1.0 | 1.7 | 1.8 | 1.8 | 1.0 |
| | (0.2) | (0.2) | (0.2) | (0.0) ^{a, b, c} | (0.1) | (0.1) | (0.2) | (0.3) ^{a, b, c} |
| Glucose (mg/dL) | 87 | 73 | 83 | 175 | 105 | 97 | 99 | 218 |
| | (7) | (16) | (7) | (35) ^{a, b, c} | (7) | (16) | (6) | (70) ^{a, b, c} |
| Phos (mg/dL) | 8.25 | 8.45 | 8.6 | 9.3 | 7.6 | 7.3 | 7.05 | 9.3 |
| | (0.75) | (0.55) | (1.0) | (3.8) | (0.7) | (0.8) | (0.75) | (5.0) |
| K (mmol/L) | 4.8 | 5.15 | 5.1 | 5.1 | 4.4 | 4.9 | 5.05 | 4.9 |
| | (0.1) | (0.18) | (0.3) | (0.6) | (0.4) | (0.4) | (0.45) | (0.65) |
| Urea N (mg/dL) | 17.5 | 16.5 | 19.0 | 28 | 20 | 20 | 21 | 20 |
| | (4.25) | (4.0) | (3.5) | (6) ^{a, b, c} | (1) | (2) | (1.8) | (3) |
| Na (mmol/L) | 145 | 144 | 144 | 140 | 145 | 143 | 142.5 | 141 |
| | (1.75) | (1.8) | (0) | (2) ^{a, b, c} | (3) | (1) | (1.0) | (2.5) ^{a} |
| T-Bil (mg/dL) | 0.00 | 0.00 | 0.00 | 0.10 | 0.00 | 0.00 | 0.00 | 0.00 |
| | (0.00) | (0.00) | (0.00) | (0.20) | (0.00) | (0.00) | (0.00) | (0.00) |
| TP (g/dL) | 5.2 | 5.5 | 5.35 | 3.7 | 5.4 | 5.8 | 5.7 | 3.90 |
| | (0.25) | (0.18) | (0.28) | (0.1) ^{a, b, c} | (0.4) | (0.5) | (0.4) | (0.85) ^{a, b, c} |
| Triglycerides | 29 | 37 | 29 | 193 | 22 | 25 | 26 | 106 |
| (mg/dL) | (12) | (22) | (5) | (111) ^{a, b, c} | (4) | (7) | (12) | (37) ^{a, b, c} |

Data represents median (IQR)
ALT, alanine aminotransferase; A/G, Albumin/Globulin ratio; ALP, Alkaline phosphatase; AST, Aspartate aminotransferase; CK, Creatine kinase; GGT, Gamma glutamyltransferase; Phos, inorganic phosphorus; K, potassium; Na, Sodium; Urea N, serum Urea nitrogen; T-Bil, total bilirubin; TP, total protein.
^{a} p < 0.05 compared with control (0 mg/kg)
^{b} p < 0.05 compared with 5 mg/kg
^{c} p < 0.05 compared with 10 mg/kg
^{d}Measurement on day of unscheduled euthanasia (Day 2), excludes animals found dead

There were no treatment-related changes in coagulation parameters in male and female rats at 5 or 10 mg/kg group at the end of treatment (Table S3) or the end of recovery (data not shown). However, at 20 mg/kg, the prothrombin time (PT) and activated partial thromboplastin time (APTT) were significantly elevated in both male and female rats, while fibrinogen levels decreased in male rats when compared to controls at the end of treatment. Of these, fibrinogen levels were outside of HCR in male rats, and both PT and APTT were outside of HCR in female rats.

TABLE 7
Coagulation data of male and female rats at the end of treatment

| | Male | | | | Female | | | |
|---|---|---|---|---|---|---|---|---|
| Parameter | 0 mg/kg | 5 mg/kg | 10 mg/kg | 20 mg/kg^{d} | 0 mg/kg | 5 mg/kg | 10 mg/kg | 20 mg/kg^{d} |
| PT | 16.3 | 16.0 | 17.5 | 21.3 | 16.3 | 16.3 | 17.1 | 23.5 |
| (seconds) | (1.6) | (0.5) | (1.4) | (1.4) ^{a, b} | (0.7) | (1.4) | (0.6) | (9.8) ^{a, b} |
| APTT | 14.1 | 14.8 | 14.6 | 18.5 | 10.5 | 10.6 | 10.9 | 29.2 |
| (seconds) | (2.9) | (1.0) | (1.1) | (3.1) ^{a, b, c} | (1.9) | (0.7) | (2.4) | (20.7) ^{a, b, c} |
| Fibrinogen | 264 | 263 | 261 | 98 | 269 | 223 | 283 | 83 |
| (mg/dL) | (50) | (9) | (55) | (19) ^{a, b, c} | (68) | (80) | (37) | (152) ^{c} |

Data represents median (IQR)
PT, Prothrombin time; APTT, Activated partial thromboplastin time.
^{a} p < 0.05 compared with control (0 mg/kg)
^{b} p < 0.05 compared with 5 mg/kg
^{c} p < 0.05 compared with 10 mg
^{d}Measurement on day of unscheduled euthanasia (Day 2), excludes animals found dead

There were no treatment-related changes in urinalysis parameters from male and female rats receiving 5 or 10 mg/kg (data not shown). Due to early removal, overnight urine samples were not collected in rats in the 20 mg/kg group and therefore could not be assessed. No treatment-related abnormalities were detected during the post-exposure ophthalmology assessments with any dosing group. In this study, treatment-related effects on the injection site could not be assessed due to surgical catheterization. Histopathological evaluations of rats in the 20 mg/kg group showed discolorations in the kidney, brain, and lungs, with bronchi and trachea filled with fluids at interim euthanasia. There were no macroscopic findings related to RTD-1 (SEQ ID NO. 1) treatment in any dose groups at interim euthanasia (for 20 mg/kg group, Day 2), terminal euthanasia (Day 8), or recovery euthanasia (Day 24). Key treatment-related microscopic findings included non-adverse, dose-dependent increased incidence of minimal to mild liver necrosis in female rats administered 5- and 10 mg/kg/day at the end of treatment. However, liver necrosis was not present in female animals (5- and 10 mg/kg/day) euthanized at the end of the recovery period and did not lead to increases in parameters included in the liver function panels (TP, albumin, total bilirubin, and liver enzymes). Therefore, these observations were considered recoverable and non-adverse. Adverse treatment-related severity ranging from minimal to moderate liver necrosis, defined by focal to multifocal areas of lytic to coagulative necrosis, was observed in male rats administered 20 mg/kg. Mild to severe adrenal necrosis, characterized by unilateral to bilaterally coagulative cortical to corticomedullar necrosis, was observed in both male and female rats administered 20 mg/kg.

Based on the lack of adverse clinical signs or abnormalities in parameters at the 10 mg/kg dose, the no-observed-adverse-effect-level (NOAEL) was established at 10 mg/kg/day in rats. The lowest observed adverse effect level (LOAEL), which the FDA defines as the lowest dose tested in preclinical species with adverse effects, was established at 20 mg/kg in rats (19).

Cynomolgus monkey studies: Overall, RTD-1 (SEQ ID NO. 1) at doses up to 15 mg/kg was well tolerated in male and female monkeys. In the non-GLP dose range-finding study, all animals survived the study without any treatment-related adverse clinical signs. The maximum tolerated dose (MTD) was established at 15 mg/kg based on these data. Similarly, in the GLP-compliant 10-day repeat dose TK study, no mortality or unscheduled euthanasia occurred at any dose level. In addition, no significant changes in body weight were observed in the monkeys. Treatment-related decrease in food consumption was observed in females at 10 mg/kg/day and in both sexes at 15 mg/kg/day during the dosing phase, but this was considered non-adverse as the animals recovered to baseline by the end of recovery and the changes in food consumption did not translate to changes in body weight or adverse clinical observations.

Non-adverse treatment-related hematological changes at the end of treatment included statistically significant, but modest increases in absolute LUC counts in male monkeys and absolute monocytes in female monkeys at 15 mg/kg when compared to the respective controls (data not shown). However, the increase in absolute LUC was resolved by the end of recovery and therefore considered non-adverse (data not shown), and the elevated absolute monocyte counts observed in female monkeys were within the HCR for female cynomolgus monkeys (20). Most notable non-adverse, but significant treatment-related changes in serum biochemical parameters included a slight reduction in inorganic phosphorus (Phos) level and an elevated glucose level in female monkeys at 15 mg/kg/day at the end of treatment. However, these changes were considered non-adverse regardless of statistical significance due to the small magnitude in change, and the elevated values were still within the HCR for female cynomolgus monkeys. A trend towards an increase in fibrinogen levels was noted in all treated monkeys when compared to the control group and baseline levels, but the changes did not reach statistical significance and returned to baseline by the end of the recovery period (data not shown). There were no treatment-related changes in urinalysis parameters or abnormalities in post-exposure ophthalmologic assessments or the electrocardiogram from cynomolgus monkeys at any dose level (data not shown). The lack of significant alterations in clinical pathology parameters in any of the animals with doses up to 15 mg/kg corroborates the results from the non-GLP dose range study in cynomolgus monkeys, which also established an MTD of 15 mg/kg based on no adverse effects on mortality, clinical observations, or bodyweight with up to 15 mg/kg intravenous RTD-1 (SEQ ID NO. 1) treatment. Due to the small number of measurements taken at the end of the recovery period, statistical analyses of the clinical pathology parameters at this time point could not be performed.

Procedure-related gross observations were recorded at the injection sites of three animals at 15 mg/kg, which included abrasions and abnormal texture likely due to repeat catheterization. However, there were no macroscopic findings related to treatment at the end of treatment or recovery. In the main group, microscopic evaluations revealed treatment-related thrombosis at the injection sites at the end of treatment, although there was a lack of a dose trend in incidence and/or severity. In the recovery group, treatment-related thrombosis at the injection site, acute inflammation, edema, hemorrhage, and fibrosis was present in animals receiving 15 mg/kg at the end of recovery. However, comparable observations were also present at the injection sites of control animals in the recovery group, which include minimal to mild injection site fibrosis and mild chronic thrombosis, suggesting that these findings are procedure-related. Lastly, minimal to mild intravascular thrombosis (thromboembolism) was observed in the lung of only the treated animals (males administered ≥10 mg/kg/day and females administered ≥5 mg/kg/day) at the end of treatment (Day 11). The thrombi within the lungs contained varying numbers of inflammatory cells both within the thrombi and in the surrounding connective tissues, and thrombi in the lungs were present predominately in mid to small arteries and capillaries in the alveolar walls with two animals. Thromboembolism was concluded to be related to the peptide administration due to the composition of the thrombi found in the lung, which were likely embolic from the thrombi formed at the injection site. These findings were also resolved by the end of the recovery period, as thrombosis was not identified in any lung sections in the control group or monkeys administered 15 mg/kg.

The summary of the NOAEL and LOAEL determined in each species is listed in Table 8. Given that the repeat administration of RTD-1 (SEQ ID NO. 1) of up to 15 mg/kg/day was well tolerated in both sexes, NOAEL was established at 15 mg/kg/day in cynomolgus monkeys. LOAEL could not be determined in cynomolgus monkeys as the highest dose examined in the GLP study was well tolerated. Based on this analysis, the HEDs that are equivalent to the NOAEL and LOAEL in rats are 3.1 mg/kg and 11.7 mg/kg for a 70 kg individual, and the HED that is equivalent to NOAEL in cynomolgus monkeys is 15.9 mg/kg for a 70 kg individual.

TABLE 8

| | Study | NOAEL | LOAEL | |
|---|---|---|---|---|
| Species | duration | (mg/kg/day) | (mg/kg/day) | Adverse effects observed at the LOAEL |
| Sprague-Dawley | 7 days | 10 | 20 | Cold to touch |
| rats | | | | Laying on side |
| | | | | Abnormal body color (pale) |
| | | | | Unable to walk |
| | | | | Extreme dehydration |
| | | | | Tremors |
| | | | | Labored respiration |
| | | | | Brain, kidney, and lung discolorations |
| | | | | Enlarged salivary glands |
| Cynomolgus | 7 days | 15 | ND | Not attained |
| monkeys | | | | |
| Cynomolgus | 10 days | 15 | ND | Not attained |
| monkeys | | | | |

### Safety

Male and female Sprague Dawley rats (n=16-21/sex) were evaluated for potential toxicity for seven day repeat administration of RTD-1 (SEQ ID NO. 1) and reversibility of any findings. Rats were divided into 3 subgroups within each dosing group: main (n=10/sex), recovery (n=θ-5/sex), and toxicokinetics (TK) (n=3-6/sex).

Mortality and clinical observations: Detailed clinical observations were recorded weekly, from a week prior to the study initiation and throughout the study including the day of necropsy in all rats. In cynomolgus monkeys, detailed clinical observations were recorded once at pre-dose and weekly following the study initiation, including the day of necropsy. All animals were observed/monitored for mortality twice daily beginning upon the arrival through release.

Body weight and food consumption: Individual body weight was recorded once at pre-dose and weekly following the initiation of dosing in all rats and three times at pre-dose and at least once weekly following the initiation of dosing in cynomolgus monkeys. Food consumption was quantitatively measured, per cage, once weekly starting on Day 1 and throughout the study in rats and assessed once daily, throughout the study in cynomolgus monkeys.

Hematology, blood chemistry, and coagulation: Blood samples for hematology, coagulation and clinical chemistry were collected from retro-orbital sinus on days of scheduled (day 8, 24, and 25 in rats belonging to the main, recovery and TK group, respectively) or unscheduled necropsy in rats. Blood samples were collected by venipuncture at pre-dose and at the end of treatment (Day 11) in cynomolgus monkeys, and additionally at the end of recovery in a few subset of male and female monkeys in the placebo (n=2/sex) and 15 mg/kg group (n=2-3/sex).

Urinalysis: Overnight urine samples were collected prior to euthanasia in rats, and at pre-dose, Day 10 and end of recovery in cynomolgus monkeys.

Ophthalmology: Ophthalmological examinations, which consisted of funduscopic (indirect ophthalmoscopy) and biomicroscopic (slit lamp) examinations, were performed at pre-dose and Day 6 in rats and at pre-dose and on Day 8 in cynomolgus monkeys.

Electrocardiogram (ECG): ECG was collected at pre-dose, Day 8 within 10 min following the end of infusion and 2 days prior to necropsy in cynomolgus monkeys.

### Methods

The pharmacokinetics and safety of intravenous (i.v.) RTD-1 (SEQ ID NO. 1) were studied in mice, rats, cynomolgus monkeys, and a vervet monkey. Lyophilized RTD-1 (SEQ ID NO. 1) (purity >98%) was dissolved in filter-sterilized saline solution and used for injections of mice and one vervet monkey. RTD-1 (SEQ ID NO. 1) solutions employing rats and cynomolgus monkeys were prepared by dilution of formulated RTD-1 (SEQ ID NO. 1) (12.5 mg/ml in 1% propylene glycol, 20 mM sodium acetate, pH 6.5) in filter-sterilized saline. The summary of study design and schedule of safety assessments are listed in Tables 9 and 10, respectively. The studies included single- and multiple-dose-ranging experiments. All protocols received local IACUC approval before the initiation of the studies.

TABLE 9
Study design of pre-clinical pharmacokinetics
and safety of intravenous RTD-1 (SEQ ID NO. 1)

| | | | | | Administration | Number of |
|---|---|---|---|---|---|---|
| Species | Study type | Regimen | Dose | | route | animals |
| Mouse | PK study | Single-dose | 5 | mg/kg | IV bolus | 24 |
| Rat | ¹⁴C-RTD-1 (SEQ | Single-dose | 5 | mg/kg | IV bolus | 5 |
| | ID NO. 1) | | | | | |
| | biodistribution | | | | | |
| | study | | | | | |
| | GLP toxicity | Single-dose | 20 | mg/kg | 20 min-IV infusion | 12 |
| | study | Multiple- | 5 mg/kg daily × | | 20 min-IV infusion | 12 |
| | | dose | 7 days | | | 12 |
| | | | 10 mg/kg daily × | | | |
| | | | 7 days | | | |
| Monkey | Non-GLP dose- | Single-dose | 0.3 | mg/kg | 1 h-IV infusion | 2 |
| | escalation study | | 1 | mg/kg | | 2 |
| | | | 3 | mg/kg | | 2 |
| | | | 10 | mg/kg | | 2 |
| | | Multiple- | 15 mg/kg daily × | | 1 h-IV infusion | 2 |
| | | dose | 7 days | | | |
| | GLP toxicity | Multiple- | 5 mg/kg daily × | | 1 h-IV infusion | 6 |
| | study | dose | 10 days | | | 6 |
| | | | 10 mg/kg daily × | | | 10 |
| | | | 10 days | | | |
| | | | 15 mg/kg daily × | | | |
| | | | 10 days | | | |
| Vervet | A pilot safety | Single dose | 3 | mg/kg | IV bolus | 1 |

study

TABLE 10
Summary of general in-life assessments and sample collections

| | A GLP 7-Day toxicity | | | | A GLP 10-day toxicity study | |
|---|---|---|---|---|---|---|
| | study in rats | | | A non-GLP PK study in | in cynomolgus monkeys | |
| Safety parameters | Main | Recovery | TK | cynomolgus monkeys | Main | Recovery^{c} |
| Individual bodyweight | Pre-dose and weekly | | | Pre-dose, Day 1 and 3 | Pre-dose and weekly | |
| Mortality | Twice daily | | | Twice daily | Twice daily | |
| Clinical observations | Weekly | | | Pre-dose, Day 1 and 3 | Pre-dose and weekly | |
| Food consumption | Once weekly | | | ND | Daily | |
| Clinical pathology^{a} | Day 8 | Day 24 | Day 25 | Day 6^{d, e}, 8^{d, e}, | Predose, | Pre-dose, Day |
| | | | | 11^{d, e}, and 15^{d, f} | Day 10 | 10 and 23/24 |
| Urinalysis | Day 8 | Day 24 | Day 25 | ND | Predose, | Pre-dose, Day |
| | | | | | Day 10 | 10 and 23/24 |
| Ophthalmology | Pre-dose, Day 6 | | | ND | Pre-dose, Day 8 | |
| Electrocardiogram | ND | | | ND | Pre-dose, | Pre-dose, Day |
| | | | | | Day 8 | 8 and 21/22 |
| Gross pathology^{b} | Day 8 | Day 24 | ND | ND | Day 11 | Day 23/24 |
| Organ weights^{b} | Day 8 | Day 24 | ND | ND | Day 11 | Day 23/24 |
| Microscopic pathology^{b} | Day 8 | Day 24 | ND | ND | Day 11 | Day 23/24 |

ND—Not determined
^{a}Includes hematology, serum chemistry, and coagulation
^{b}Any animals found dead or pre-terminally euthanized are assessed at the time of necropsy
^{c}Includes a subset of cynomolgus monkeys in the main group that received 0 mg/kg (placebo) or 15 mg/kg of intravenous RTD-1 (SEQ ID NO. 1)
^{d}Includes cynomolgus monkeys that received seven (7) daily doses of 15 mg/kg of intravenous RTD-1 (SEQ ID NO. 1)
^{e}Blood collected for hematology and serum chemistry
^{f}Blood collected for coagulation

### Pharmacokinetics

Mouse studies: All procedures and protocols involving the use of animals were reviewed and approved by the University of Southern California (USC) IACUC (protocol #20538). Briefly, male (31.7-37.7 g) and female (25.2-35.6 g) CD-1 mice (Charles River Laboratories) were administered a single 5 mg/kg i.v. bolus injection of RTD-1 (SEQ ID NO. 1) into the lateral tail vein. A total of 24 mice were separated into six groups (n=2/sex/group), with each group assigned to a single, pre-determined time point. Blood samples were collected at 0.25, 1, 2, 4, 8, and 24 h post-dose via terminal cardiac puncture. The collected samples were centrifuged to separate the plasma and stored at −80° C. until analysis.

Rat studies: Pharmacokinetics of RTD-1 (SEQ ID NO. 1) in Sprague-Dawley rats was evaluated as part of a Good Lab Practice (GLP) 7-day toxicity study. The study protocol was reviewed and approved by the Citoxlab USA IACUC and was conducted in accordance with guidelines from the USA National Research Council. On the day of the dosing (Day 1), male (229-272 g) and female (186-214 g) rats (n=6/sex/group) were assigned to receive repeated doses of 0 (placebo), 5,10 or 20 mg/kg of RTD-1 (SEQ ID NO. 1) once daily for 7 days via i.v. infusion (20 min ±3 min). The intravenous route was selected as this is the intended route of administration for prophylactic treatment of microbial infection. The doses were chosen based on a pilot single dose-escalation study in rats, which established the MTD of 20 mg/kg (data not shown). Two subgroups of rats with alternating blood sampling schemes (n=3/sex/group) were assigned as follows: subgroup A with blood collections at 0 (pre-dose), 0.5, 6, and 24 h post-infusion and subgroup B with blood collections at 0.083, 2, and 12 h post-infusion. The 24 h post-infusion samples on Day 1 were taken before the administration of the dose on Day 2. Serial blood samples were collected into K2EDTA tubes at the above-mentioned time points on Days 1 and 7 and once on Day 25 (recovery). The samples were centrifuged at 2,700 g for 10 min at 5° C. to separate plasma from the blood and stored at −80° C. until analysis.

Cynomolgus monkey studies: A non-GLP dose range-finding PK study and a GLP 10-day toxicity study were conducted in cynomolgus monkeys (Macacafascicularis). The study protocol was reviewed and approved by the Citoxlab USA IACUC and was conducted in accordance with guidelines from the USA National Research Council. The non-GLP dose range-finding study included a single ascending dose and a multiple-dose evaluation. In the single ascending dose PK study, male (3.49-3.57 kg) and female (2.66-2.79 kg) cynomolgus monkeys (n=1/sex/group) were randomly assigned to one of two dose groups. Group 1 received a single dose of 0.3 mg/kg RTD-1 (SEQ ID NO. 1) as an i.v. infusion (60 min ±5 min) on Day 1 and a single dose of 3 mg/kg RTD-1 (SEQ ID NO. 1) on Day 3, while Group 2 received a single dose of 1 mg/kg RTD-1 (SEQ ID NO. 1) on Day 1 and a single dose of 10 mg/kg RTD-1 (SEQ ID NO. 1) on Day 3. The doses used in this study are based on a pilot study in cynomolgus monkeys, which demonstrated the safety of single i.v. doses up to and including 10 mg/kg (data not shown). Blood samples were collected into K₂EDTA tubes at the following time points: pre-dose and approximately 0.083, 0.25, 0.5, 1, 2, 4, 8, 12, and 24 h post end of infusion, on Days 1 and 3. In the multiple-dose study, repeated doses of 15 mg/kg RTD-1 (SEQ ID NO. 1) were administered once daily for seven days via i.v. infusion (60 min ±5 min) (n=1 per sex). Serial blood samples were collected into K₂EDTA at pre-dose, and at approximately 0.083, 0.25, 0.5, 1, 2, 4, 8, 12, and 24 h post end of infusion, on Days 1, 4, and 7. The 24 h post-infusion samples on Days 1 and 4 were taken before the administration of the dose on Days 2 and 5, respectively. In the GLP 10-day toxicity study, PK was evaluated following multiple ascending doses of RTD-1 (SEQ ID NO. 1). Male (2.52-3.65 kg) and female (2.39-3.18 kg) cynomolgus monkeys (n=3-5/sex/group) received placebo (sterile saline) or 5, 10 or 15 mg/kg of RTD-1 (SEQ ID NO. 1) once daily for 10 days via i.v. infusion (60 min ±5 min). Serial blood samples were collected into K₂EDTA tubes at the following time points: pre-dose, 0.083, 0.5, 2, 6, and 12 h post-infusion on Day 1 and pre-dose, 0.083, 0.5, 2, 6, 12, and 24 h post-infusion on Day 10. Additional blood samples were collected on Days 12 and 24 from monkeys that received 0 or 15 mg/kg (n=2/sex/group). All samples were centrifuged at ˜2700× g for 10 min at −5° C. to isolate plasma and stored at −80° C. until analysis.

Vervet monkey studies: As a pilot safety study, a single dose of RTD-1 (SEQ ID NO. 1) at 3 mg/kg was administered to an adult male vervet/African green monkey (Chlorocebus aethiops sabaeus) via i.v. bolus. Serial blood samples were collected at 0.5, 1, 4, 8, 24, 48, 72, 96, 120, and 192 h post-administration.

Bioanalytical analysis: Clarified plasma samples from mice were directly diluted into 5% formic acid/5% acetonitrile. Quantitative analysis of RTD-1 (SEQ ID NO. 1) was performed by LC-MS/MS with reverse-phase liquid chromatography (XBridge BEH phenyl 3.5 μm 3×100 mm column, Waters #186003328) on an Acquity H-Class UPLC (Waters) coupled to a Xevo TQ-S tandem electrospray mass spectrometry running MassLynx V4.1 (Waters). Quantitative mass spectroscopy was performed by multiple-reaction monitoring transition 417.32 >517.21, with the area under the curve determined by TargetLynx (Waters). A synthetic theta defensin-like peptide was used as an internal standard (IS). The lower limit of quantification (LLOQ) of the assay was 1 ng/mL. Intra- and inter-assay precision (percent coefficient of variation [CV]) was <3%, and intra and interassay accuracy (% relative error) was <5%. Plasma RTD-1 (SEQ ID NO. 1) concentration analyses for rats and cynomolgus monkey studies were performed at MicroConstants (San Diego, CA) by HPLC using a Mac-Mod Analytical ACE C4 column. The mobile phase was nebulized using heated nitrogen in Z-Spray source/interface set to electrospray positive ionization mode, and the compound was detected using MS/MS (LLOQ=10.0 ng/mL). Details of the method are summarized in the MicroConstants method No. MN20038. Clarified plasma samples from a serially sampled vervet were quantified by ultra-performance liquid chromatography Waters Acquity H-Class UPLC. The plasma samples were diluted directly (1:10) into 5% formic acid/5% acetonitrile and quantified by photodiode array (PDA AUC of 210 nm) using (C18 XBridge BEH 2.5 μm 2.1×150 mm Waters #186006709) running Empower software. RTD-1 (SEQ ID NO. 1) peak and mass confirmation was performed on post PDA eluent using a Micromass Quattro Ultima mass spectrometer with MassLynx 4.1 (Waters). The lower limit of quantitation (LLOQ) ranged from 10-30 ng/mL (determined by sample background) to a upper limit of 50 μg/mL. RTD-2 (10 ug/mL) was used as an internal standard. Intra- and interassay precision (% coefficient of variation [CV]) was <3%, and intra and interassay accuracy (% relative error) was <5%.

Pharmacokinetic analysis: Non-compartmental analysis (NCA) was performed using Phoenix® WinNonlin (version 8.3.1, Certara USA, Inc.; Princeton NJ) to determine the PK parameters in mice, rats, cynomolgus monkeys, and the vervet. Nominal sampling times were used in the analysis, and data below the lower limit of quantification of the assay were excluded from the analysis. The maximum plasma concentration (Cmax) was determined from visual inspection of the data. In addition, the following parameters were calculated: terminal elimination rate constant (Xz), area under the curve extrapolated to infinity (AUC0-∞), area under the curve to dosing interval (AUCT), mean residence time (MRT), clearance (CL), and volume of distribution at steady state (V_{SS}). AUC was calculated using the linear up, log down method, and the Xz was calculated using up to the last four data points of the log-linear terminal phase of the concentration-time profile. Due to sparsely sampled data in mice and rats, the sparse sampling calculation methodology in Phoenix WinNonlin was used, which generated a single estimate without standard error for all parameters, except for Cmax. For rats and cynomolgus monkeys, all parameters were calculated using sampling times relative to the beginning of the i.v. infusion.

Dose proportionality: Dose proportionality of Cmax and AUC0-∞ was evaluated in cynomolgus monkeys administered a single i.v. dose of RTD-1 (SEQ ID NO. 1) ranging from 0.3 to 15 mg/kg using a natural log-transformed power model (37). Dose proportionality was concluded if the slope and the corresponding 95% confidence interval of the linear regression included.

Interspecies allometric scaling: Single-dose PK data from mice, rats, cynomolgus monkeys, and a vervet was used to predict human PK parameters using simple allometry. The relationship between CL or V_{SS} obtained from the NCA, and the body weight was described using the following equation: Y=a·BW^{b}, where Y is the PK parameter (e.g., CL or V_{SS}), BW is the bodyweight of the species, a is an allometric coefficient, and b is an allometric exponent (34, 38). Linear regression was performed on log-transformed data. The predicted human equivalent dose was calculated based on the allometrically scaled clearance using the following equation: Dose=CL AUC_{0-∞}. The corresponding average AUCτ and AUC_{0-∞} at NOAEL and LOAEL, respectively, were used to convert the doses at NOAEL and LOAEL in preclinical animals to HED.

Biodistribution [¹⁴C]-radiolabeled RTD-1 (SEQ ID NO. 1) in female rats: Five SD rats (195 and 200 g b.w.) with jugular vein catheters (JVC) were each injected with 200 μL of 5 mg/mL RTD-1 (SEQ ID NO. 1) in saline containing ˜4 million CPM of [¹⁴C]-RTD-1 (SEQ ID NO. 1). The ¹⁴C-RTD-1 (SEQ ID NO. 1) was created by substituting the natural glycine residue on position 1 of the cyclic peptide with a ¹⁴C labeled glycine. The JVC line was cleaned with 70% isopropyl alcohol, and the line plug was removed. A new 25G blunt needle with a 1 mL syringe containing injectable solutions was used for each injection. The JVC line was first cleared with 100 μL saline, followed by the RTD-1 (SEQ ID NO. 1) solution, then cleared with an additional 100 μL of saline. Tissues and organs were harvested into separated vials and weighed. Small organs such as lymph nodes, kidneys, and heart/lungs were processed whole.

Large organs (e.g., liver, muscles, subcutaneous fat pads) were sampled from representative areas or those of interest (e.g., subcutis at the injection site). Organs were then dissolved in 2 mL SOLVABLE (Perkin Elmer 6NE9100) for up to 1 g of tissue. For a large section of skin and other organs, 4 or 6 mL were added. Vials were incubated in a 60° C. water bath for 18-22 hours, then removed and allowed to cool to room temperature. Two mL of dissolved tissue were added to a fresh scintillation tube for color correction with 100 μL of 0.1 M EDTA and 2×100 μL 30% hydrogen peroxide. Samples were allowed to stand at room temperature for 1 hour, incubated in a 37° C. incubator for 1 hour, then incubated in a 60° C. water bath for 1 hour. If necessary to prevent boiling over, samples were removed from the heat source temporarily before continuing. Samples were cooled before 10 mL of Ultima Gold (Perkin Elmer 6013321) were added to each vial, then contents were mixed and allowed to stand in the dark at 22° C. for 1 h. Scintillation counting was an average of 2×1-minute counts using a Packard TRI-CARD 2100TR scintillation counter. Urine was collected over 1 h or 24 h after i.v. infusion and 500 μL was added to a scintillation vial processed as described above for scintillation counting. The stomach and its content were dissolved with 6 mL of SOLVABLE and processed as other tissues. The duodenum, jejunum, and ileum were flushed with saline to remove luminal contents then the tissues were processed with SOLVABLE as described above. The large intestine was opened lengthwise to remove fecal content and rinsed with saline to remove remaining luminal contents, then processed as described above. Feces were collected, transferred into a 500 mL plastic cup, and treated with 50 mL of 7% sodium hypochlorite and allowed to react for 1 hour at 22° C. followed by 1 h in a 60° C. water bath. Two ml of the resulting suspension were transferred to a scintillation vial and mixed with 10 mL of scintillation fluid. Contents were mixed and allowed to stand for 1 hour before scintillation counting.

### Safety

Evaluation of safety in rats and cynomolgus monkeys was based on clinical observations, survival, body weight, food consumption, clinical pathology (hematology, clinical chemistry, and coagulation), urinalysis, ophthalmology, macroscopic findings at necropsy, and microscopic histopathology. The schedule of assessments for these studies is summarized in Table 2.

### Statistical Analysis

Statistical analysis of the PK data was performed with GraphPad Prism version 9.1.2 (GraphPad Software, Inc., San Diego, CA). Shapiro-Wilk test was used to check for normality.

One-way ANOVA with Bonferroni's multiple comparisons test was performed to compare Cmax across doses (5-, 10- and 20 mg/kg) in rats, and λz, AUC, Cmax, CL, and V_{SS} across doses (5-, 10- and 15 mg/kg) in cynomolgus monkeys. Unpaired t-test was used to compare PK parameters (λz, AUC, Cmax, CL, and V_{SS}) between different days (Days 1 vs. 10) within each dosing group and between sexes. Statistical analyses of safety data were performed using SAS 9.4, considering a 95% statistical significance. Differences in body weight, change in body weight, and clinical pathology (hematology, serum chemistry, coagulation) were compared between each dose group using Kruskal Wallis with Dunn's multiple comparison test. The data analysis was performed independently for each sex.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refer to at least one of something selected from the group consisting of A, B, C . . . and N, the text should be interpreted as requiring only one element from the group, not A plus N, or B plus N, etc.

What is claimed is:

1. A method of providing prophylactic treatment for infection by a microbe, comprising
identifying an individual in need of prophylactic treatment for the microbial infection; and
administering an effective amount of the effective θ-defensin or the effective θ-defensin analog to the individual in need of treatment, wherein the effective amount provides antimicrobial activity directed to the microbe in at least 50% of peak plasma concentration of the effective θ-defensin or the effective θ-defensin analog.

2. The method of claim 1, further comprising identifying an effective θ-defensin or an effective θ-defensin analog that maintains at least 50% of peak plasma concentration of the effective θ-defensin or an effective θ-defensin analog for at least 4 hours following administration.

3. The method of claim 1 or claim 2, wherein the effective θ-defensin is selected from the group consisting of RTD-1 (SEQ ID NO. 1), RTD-2 (SEQ ID NO. 2), RTD-3 (SEQ ID NO. 3), RTD-4 (SEQ ID NO. 4), RTD-5 (SEQ ID NO. 5), RTD-6 (SEQ ID NO. 6), BTD-1 (SEQ ID NO. 7), BTD-2 (SEQ ID NO. 8), BTD-3 (SEQ ID NO. 9), BTD-4 (SEQ ID NO. 10), BTD-5 (SEQ ID NO. 11), BTD-6 (SEQ ID NO. 12), BTD-7 (SEQ ID NO. 13), BTD-8 (SEQ ID NO. 14), BTD-9 (SEQ ID NO. 15), and/or BTD-10 (SEQ ID NO. 16), and a peptide derived from HTDp (SEQ ID NO. 17).

4. The method of claim 1 or claim 2, wherein the effective θ-defensin analog is selected from the group consisting of peptide 1 (SEQ ID NO. 18), peptide 2 (SEQ ID NO. 19), peptide 3 (SEQ ID NO. 20), peptide 4 (SEQ ID NO. 21), peptide 5 (SEQ ID NO. 22), peptide 6 (SEQ ID NO. 23), peptide 7 (SEQ ID NO. 24), peptide 8 (SEQ ID NO. 25), peptide 9 (SEQ ID NO. 26), peptide 10 (SEQ ID NO. 27), peptide 11 (SEQ ID NO. 28), peptide 12 (SEQ ID NO. 29), peptide 13 (SEQ ID NO. 30), peptide 14 (SEQ ID NO. 31), and peptide 15 (SEQ ID NO. 32).

5. The method of one of claims 1 to 4, wherein the individual is further identified as a high-risk individual.

6. The method of claim 5, wherein the high risk individual is identified as a neonate, a premature infant, a child with an under or partially developed immune system, an elderly individual, as recovering from surgery, as recovering from accidental injury, as having cancer, as immunocompromised, as having undergone organ, bone marrow, or stem cell transplantation, individuals receiving or recovering from chemotherapy, as receiving or recovering from radiotherapy, as receiving or recovering from immunotherapy, as receiving or recovering from immunosuppression therapy, as having underlying heart disease and/or damage, as having underlying kidney disease and/or damage, as having underlying liver disease and/or damage, as having underlying lung disease and/or damage, or as having obesity.

7. The method of claim 5, wherein the high risk individual is identified on the basis of exposure to others that are within a distance over which the microbe can be transmitted.

8. The method of one of claims 1 to 7, wherein the microbe is a virus.

9. The method of claim 6, wherein the virus is SARS-CoV-2.

10. The method of one of claims 1 to 7, wherein the microbe is a bacterium.

11. The method of one of claims 1 to 7, wherein the microbe is a fungus.

12. The method of one of claims 1 to 7, wherein the microbe is a protozoan.

13. The method of one of claims 1 to 12, wherein the effective θ-defensin is RTD-1 (SEQ ID NO. 1).

14. The method of one of claims 1 to 13, wherein the effective θ-defensin or effective θ-defensin analog is administered at up to 15 mg/kg.

15. The method of one of claims 1 to 14, wherein administering comprises a single administration of the effective θ-defensin or effective θ-defensin analog.

16. The method of claim 15, wherein the single administration comprises an amount of the effective θ-defensin or effective θ-defensin analog to provide from 0.1 mg/kg to 15 mg/kg to the individual in need of treatment.

17. The method of one of claims 1 to 14, wherein administering comprises a plurality of administrations of the effective θ-defensin or effective θ-defensin analog.

18. The method of claim 17, wherein the protocol comprises an administration frequency of once every 12 hours to once every 48 hours.

19. The method of claim 17 or 18, wherein each administration comprises an amount of the effective θ-defensin or effective θ-defensin analog sufficient to provide from 0.1 mg/kg to 15 mg/kg to the individual in need of treatment.

20. The method of one of claims 17 to 19, wherein the plurality of administrations comprises administration of a first amount of the effective θ-defensin or effective θ-defensin analog and administration of a second amount of the effective θ-defensin or effective θ-defensin analog

21. Use of a formulation comprising a θ-defensin or a θ-defensin analog for prophylactic treatment for infection by a microbe, wherein the formulation comprises an effective θ-defensin or an effective θ-defensin analog that is identified as maintaining at least 50% of peak plasma concentration of the effective θ-defensin or an effective θ-defensin analog for at least 4 hours following administration, wherein the formulation provide the effective θ-defensin or an effective θ-defensin analog in an amount sufficient to provide an antimicrobial effect for at least 4 hours.

22. The use of claim 21, wherein the effective θ-defensin is selected from the group consisting of RTD-1 (SEQ ID NO. 1), RTD-2 (SEQ ID NO. 2), RTD-3 (SEQ ID NO. 3), RTD-4 (SEQ ID NO. 4), RTD-5 (SEQ ID NO. 5), RTD-6 (SEQ ID NO. 6), BTD-1 (SEQ ID NO. 7), BTD-2 (SEQ ID NO. 8), BTD-3 (SEQ ID NO. 9), BTD-4 (SEQ ID NO. 10), BTD-5 (SEQ ID NO. 11), BTD-6 (SEQ ID NO. 12), BTD-7 (SEQ ID NO. 13), BTD-8 (SEQ ID NO. 14), BTD-9 (SEQ ID NO. 15), and/or BTD-10 (SEQ ID NO. 16), and a peptide derived from HTDp (SEQ ID NO. 17).

23. The use of claim 21, wherein the effective θ-defensin analog is selected from the group consisting of peptide 1 (SEQ ID NO. 18), peptide 2 (SEQ ID NO. 19), peptide 3 (SEQ ID NO. 20), peptide 4 (SEQ ID NO. 21), peptide 5 (SEQ ID NO. 22), peptide 6 (SEQ ID NO. 23), peptide 7 (SEQ ID NO. 24), peptide 8 (SEQ ID NO. 25), peptide 9 (SEQ ID NO. 26), peptide 10 (SEQ ID NO. 27), peptide 11 (SEQ ID NO. 28), peptide 12 (SEQ ID NO. 29), peptide 13 (SEQ ID NO. 30), peptide 14 (SEQ ID NO. 31), and peptide 15 (SEQ ID NO. 32).

24. The use of one of claims 21 to 23, wherein the microbe is a virus.

25. The use of claim 24, wherein the virus is SARS-CoV-2.

26. The use of one of claims 21 to 23, wherein the microbe is a bacterium.

27. The use of one of claims 21 to 23, wherein the microbe is a fungus.

28. The use of one of claims 21 to 23, wherein the microbe is a protozoan.

29. The use of one of claims 21 to 28, wherein the effective θ-defensin is RTD-1 (SEQ ID NO. 1).

30. The use of one of claims 21 to 29, wherein the formulation provides the effective θ-defensin or effective θ-defensin analog at up to 15 mg/kg.

31. The use of one of claims 21 to 30 wherein the formulation is formulated to provide prophylaxis following a single administration of the effective θ-defensin or effective θ-defensin analog.

32. The use of one of claims 21 to 30, wherein the formulation is formulated to provide prophylaxis following a plurality of administrations of the effective θ-defensin or effective θ-defensin analog.

33. The use of claim 32, wherein the formulation is formulated to provide at each of the plurality of administrations an amount of the effective θ-defensin or effective θ-defensin analog sufficient to provide from 0.1 mg/kg to 15 mg/kg.

34. A formulation for providing prophylactic treatment of an infection by a microbe, comprising a θ-defensin or a θ-defensin analog for prophylactic treatment for infection by a microbe, wherein the formulation comprises an effective θ-defensin or an effective θ-defensin analog that is identified as maintaining at least 50% of peak plasma concentration of the effective θ-defensin or an effective θ-defensin analog for at least 4 hours following administration, wherein the formulation provide the effective θ-defensin or an effective θ-defensin analog in an amount sufficient to provide an antimicrobial effect for at least 4 hours.

35. The formulation of claim 34, wherein the effective θ-defensin is selected from the group consisting of RTD-1 (SEQ ID NO. 1), RTD-2 (SEQ ID NO. 2), RTD-3 (SEQ ID NO. 3), RTD-4 (SEQ ID NO. 4), RTD-5 (SEQ ID NO. 5), RTD-6 (SEQ ID NO. 6), BTD-1 (SEQ ID NO. 7), BTD-2 (SEQ ID NO. 8), BTD-3 (SEQ ID NO. 9), BTD-4 (SEQ ID NO. 10), BTD-5 (SEQ ID NO. 11), BTD-6 (SEQ ID NO. 12), BTD-7 (SEQ ID NO. 13), BTD-8 (SEQ ID NO. 14), BTD-9 (SEQ ID NO. 15), and/or BTD-10 (SEQ ID NO. 16), and a peptide derived from HTDp (SEQ ID NO. 17).

36. The formulation use of claim 34, wherein the effective θ-defensin analog is selected from the group consisting of peptide 1 (SEQ ID NO. 18), peptide 2 (SEQ ID NO. 19), peptide 3 (SEQ ID NO. 20), peptide 4 (SEQ ID NO. 21), peptide 5 (SEQ ID NO. 22), peptide 6 (SEQ ID NO. 23), peptide 7 (SEQ ID NO. 24), peptide 8 (SEQ ID NO. 25), peptide 9 (SEQ ID NO. 26), peptide 10 (SEQ ID NO. 27), peptide 11 (SEQ ID NO. 28), peptide 12 (SEQ ID NO. 29), peptide 13 (SEQ ID NO. 30), peptide 14 (SEQ ID NO. 31), and peptide 15 (SEQ ID NO. 32).

37. The formulation of one of claims 34 to 36, wherein the microbe is a virus.

38. The formulation of claim 37, wherein the virus is SARS-CoV-2.

39. The formulation of one of claims 34 to 36, wherein the microbe is a bacterium.

40. The formulation of one of claims 34 to 36, wherein the microbe is a fungus.

41. The formulation of one of claims 34 to 36, wherein the microbe is a protozoan.

42. The formulation of one of claims 34 to 41, wherein the effective θ-defensin is RTD-1 (SEQ ID NO. 1).

43. The formulation of one of claims 34 to 42, wherein the formulation is formulated to provide the effective θ-defensin or effective θ-defensin analog at up to 15 mg/kg.

44. The formulation of one of claims 34 to 43, wherein the formulation is formulated to provide prophylaxis following a single administration of the effective θ-defensin or effective θ-defensin analog.

45. The formulation of one of claims 34 to 43, wherein the formulation is formulated to prophylaxis following a plurality of administrations of the effective θ-defensin or effective θ-defensin analog.

46. The formulation of claim 45, wherein the formulation is formulated to provide at each of the plurality of administrations an amount of the effective θ-defensin or effective θ-defensin analog sufficient to provide from 0.1 mg/kg to 15 mg/kg.

47. Use of a θ-defensin or θ-defensin analog in preparing a formulation for providing prophylactic treatment of an infection by a microbe, comprising a θ-defensin or a θ-defensin analog for prophylactic treatment for infection by a microbe, wherein the formulation comprises an effective θ-defensin or an effective θ-defensin analog that is identified as maintaining at least 50% of peak plasma concentration of the effective θ-defensin or an effective θ-defensin analog for at least 4 hours following administration, wherein the formulation provide the effective θ-defensin or an effective θ-defensin analog in an amount sufficient to provide an antimicrobial effect for at least 4 hours.

48. The use of claim 47, wherein the effective θ-defensin is selected from the group consisting of RTD-1 (SEQ ID NO. 1), RTD-2 (SEQ ID NO. 2), RTD-3 (SEQ ID NO. 3), RTD-4 (SEQ ID NO. 4), RTD-5 (SEQ ID NO. 5), RTD-6 (SEQ ID NO. 6), BTD-1 (SEQ ID NO. 7), BTD-2 (SEQ ID NO. 8), BTD-3 (SEQ ID NO. 9), BTD-4 (SEQ ID NO. 10), BTD-5 (SEQ ID NO. 11), BTD-6 (SEQ ID NO. 12), BTD-7 (SEQ ID NO. 13), BTD-8 (SEQ ID NO. 14), BTD-9 (SEQ ID NO. 15), and/or BTD-10 (SEQ ID NO. 16), and a peptide derived from HTDp (SEQ ID NO. 17).

49. The use of claim 47, wherein the effective θ-defensin analog is selected from the group consisting of peptide 1 (SEQ ID NO. 18), peptide 2 (SEQ ID NO. 19), peptide 3 (SEQ ID NO. 20), peptide 4 (SEQ ID NO. 21), peptide 5 (SEQ ID NO. 22), peptide 6 (SEQ ID NO. 23), peptide 7 (SEQ ID NO. 24), peptide 8 (SEQ ID NO. 25), peptide 9 (SEQ ID NO. 26), peptide 10 (SEQ ID NO. 27), peptide 11 (SEQ ID NO. 28), peptide 12 (SEQ ID NO. 29), peptide 13 (SEQ ID NO. 30), peptide 14 (SEQ ID NO. 31), and peptide 15 (SEQ ID NO. 32).

50. The use of one of claims 47 to 49, wherein the microbe is a virus.

51. The use of claim 50, wherein the virus is SARS-CoV-2.

52. The use of one of claims 47 to 49, wherein the microbe is a bacterium.

53. The use of one of claims 47 to 49, wherein the microbe is a fungus.

54. The use of one of claims 47 to 49, wherein the microbe is a protozoan.

55. The use of one of claims 47 to 54, wherein the effective θ-defensin is RTD-1 (SEQ ID NO. 1).

56. The use of one of claims 47 to 55, wherein the formulation is formulated to provide the effective θ-defensin or effective θ-defensin analog at up to 15 mg/kg.

57. The use of one of claims 46 to 56 wherein the formulation is formulated to provide prophylaxis following a single administration of the effective θ-defensin or effective θ-defensin analog.

58. The use of one of claims 47 to 56, wherein the formulation is formulated to provide prophylaxis following a plurality of administrations of the effective θ-defensin or effective θ-defensin analog.

59. The use of claim 58, wherein the formulation is formulated to provide at each of the plurality of administrations an amount of the effective θ-defensin or effective θ-defensin analog sufficient to provide from 0.1 mg/kg to 15 mg/kg.


SEQUENCE LISTING (32 sequences)

SEQ ID NO: 1 - protein, 16 aa, Macaca mulatta
GFCRCLCRRGVCRCIC

SEQ ID NO: 2 - protein, 16 aa, Macaca mulatta
GVCRCLCRRGVCRCLC

SEQ ID NO: 3 - protein, 16 aa, Macaca mulatta
GFCRCICTRGFCRCIC

SEQ ID NO: 4 - protein, 16 aa, Macaca mulatta
GICRCICTRGFCRCIC

SEQ ID NO: 5 - protein, 16 aa, Macaca mulatta
GICRCLCRRGVCRCIC

SEQ ID NO: 6 - protein, 16 aa, Macaca mulatta
GICRCICVLGICRCIC

SEQ ID NO: 7 - protein, 16 aa, Papio anubis
CVCRRGVCRCVCTRGF

SEQ ID NO: 8 - protein, 32 aa, Papio anubis
CVCRRGVCRCVCRRGVCVCRRGVCRCVCRRGV

SEQ ID NO: 9 - protein, 16 aa, Papio anubis
CICLLGICRCVCTRGF

SEQ ID NO: 10 - protein, 16 aa, Papio anubis
CICLLGICRCVCTRGF

SEQ ID NO: 11 - protein, 16 aa, Papio anubis
CICLLGICRCVCRRGV

SEQ ID NO: 12 - protein, 16 aa, Papio anubis
CICLLGICRCICLLGI

SEQ ID NO: 13 - protein, 16 aa, Papio anubis
CFCRRGVCRCVCTRGF

SEQ ID NO: 14 - protein, 16 aa, Papio anubis
CFCRRGVCRCVCRRGV

SEQ ID NO: 15 - protein, 16 aa, Papio anubis
CFCRRGVCRCICLLGI

SEQ ID NO: 16 - protein, 16 aa, Papio anubis
CFCRRGVCRCFCFFGV

SEQ ID NO: 17 - protein, 76 aa, Homo sapiens
MRTFALLTHAQAEPLQARADEAAAAMLLLVALQEQPGADDQEMAHAFTWHESAALPLSDSARGLRCICGRGICRLL

SEQ ID NO: 18 - protein, 13 aa, synthetic construct
GFCRCRRGVCRCT

SEQ ID NO: 19 - protein, 14 aa, synthetic construct
GVCIVRRRFCLCRR

SEQ ID NO: 20 - protein, 14 aa, synthetic construct
GVCLCIRGRCRCRR

SEQ ID NO: 21 - protein, 14 aa, synthetic construct
CICRRRVCICGRGV

SEQ ID NO: 22 - protein, 14 aa, synthetic construct
CICRRRFCLCRRGV

SEQ ID NO: 23 - protein, 14 aa, synthetic construct
CLCRRGVCLCRRGV

SEQ ID NO: 24 - protein, 14 aa, synthetic construct
CICRRGVCICRRGV

SEQ ID NO: 25 - protein, 14 aa, synthetic construct
CACARRFCACRRGV

SEQ ID NO: 26 - protein, 14 aa, synthetic construct
CSCRRRFCICRRGV

SEQ ID NO: 27 - protein, 14 aa, synthetic construct
CICRRRFCSCRRGV

SEQ ID NO: 28 - protein, 14 aa, synthetic construct
CSCRRRFCLCRRGV

SEQ ID NO: 29 - protein, 14 aa, synthetic construct
CICRRRFCLCRRGA

SEQ ID NO: 30 - protein, 14 aa, synthetic construct
CACRRRFCACRRGV

SEQ ID NO: 31 - protein, 14 aa, synthetic construct
CICRRRVCICRRGV

SEQ ID NO: 32 - protein, 14 aa, synthetic construct
CICRRRACLCRRGL